(19) United States
(12) Patent Application Publication
(10) Pub. No.: US 2014/0243396 A1
(43) Pub. Date: Aug. 28, 2014

(54) METHOD OF TREATING MUCOEPIDERMOID CARCINOMA

(71) Applicant: DANA-FARBER CANCER INSTITUTE, INC., Boston, MA (US)

(72) Inventors: James D. Griffin, Needham, MA (US); Lizi Wu, Gainesville, FL (US); Jie Chen, San Diego, CA (US)

(21) Appl. No.: 14/347,961

(22) PCT Filed: Sep. 27, 2012
(86) PCT No.: PCT/US2012/057480

§ 371 Date: Mar. 27, 2014

Related U.S. Application Data
(60) Provisional application No. 61/541,758, filed on Sep. 30, 2011.
(60) Provisional application No. 61/660,377, filed on Jun. 15, 2012.

(51) Int. Cl. C07D 471/04; A61K 45/06; A61K 31/352; A61K 31/7088; A61K 31/4745; A61K 31/4015

(52) U.S. Cl. CPC C07D 471/04; A61K 31/4745; A61K 31/4015; A61K 31/352; A61K 31/7088; A61K 45/06
USPC 514/44 A; 514/293; 514/424; 514/455

(57) ABSTRACT

Imidazoquinolines, as set forth in formula (I), are useful for inhibiting growth or proliferation of mucoepidermoid carcinoma cells. The therapeutic and prophylactic treatments provided by this invention are practiced by administering to a patient in need thereof an amount of a compound of formula (I) that is effective to inhibit growth or proliferation of the mucoepidermoid carcinoma cells.

### RELATED APPLICATIONS

This application claims the benefit of the filing date under 35 U.S.C. §119(e) of the following Provisional U.S. patent application Ser. Nos. 61/660,377 filed Jun. 15, 2012, and 61/541,758, filed Sep. 30, 2011, which are hereby incorporated by reference in their entirety.

### FEDERALLY SPONSORED RESEARCH OR DEVELOPMENT

This invention was made with government support under federal grant number CA-66996 awarded by National Institutes of Health. The government has certain rights in the invention.

### BACKGROUND OF THE INVENTION

Mucoepidermoid carcinomas (MEC) are the most common malignant salivary gland tumors and the second most frequent lung tumors of bronchial gland origin. In addition, MEC have been reported to occur in the trachea, esophagus, breast, pancreas, cervix and thyroid gland. Systemic treatment of metastatic MEC tumors has been disappointing.

There is a fusion oncogene that involves a t(11; 19)(q21; p13) translocation in salivary gland MECs; Tonon, G., Modi, S., Wu, L., Kubo, A., Coxon, A. B., Komiya, T., O'Neil, K., Stover, K., El-Naggar, A., Griffin, J. D., Kirsch, I. R., and Kaye, F. J.; t(11;19)(q21;p13) translocation in mucoepidermoid carcinoma creates a novel fusion product that disrupts a Notch signaling pathway. Nat Genet, 2003, 33: 208-213. This translocation is detected in up to 80% of all MECs, and also in some benign tumors, including Warthin's tumors and clear cell hidradenoma of the skin. The translocation encodes a fusion protein, termed CRTC1-MAML2, which consists of 42 amino acids of the N-terminal CREB (cAMP Response Element Binding Protein)-binding domain of the CREB regulator CRTC1 and 981 amino acids from the C-terminal transactivation domain (TAD) of Notch co-activator MAML2. MAML2 is a member of the Notch co-activator mastermind family proteins and is required for Notch signaling. CRTC1 (also known as MECT1, TORC1 or WAMTP1) belongs to a family of conserved CREB co-activators (14, 15). Binding of CRTC1 to CREB enhances recruitment of TAFII130 to the CREB complex, and thus activates downstream signaling.

There is abundant evidence that CRTC1-MAML2 is an oncogene. For example, ectopic expression of CRTC1-MAML2, but not over-expression of MAML2, induced foci formation. Tonon et al., supra. In addition, injection of CRTC1-MAML2 transfected RK3E cells into nude mice caused tumor formation in vivo and sustained expression of the fusion was required for tumorigenesis in these mice; Komiya, T., Park, Y., Modi, S., Coxon, A. B., Oh, H., and Kaye, F. J.; Sustained expression of Mect1-Mam12 is essential for tumor cell growth in salivary gland cancers carrying the t(11;19) translocation. Oncogene, 2006, 25: 6128-6132. It was reported that CRTC1-MAML2 activates multiple cAMP/CREB genes by constitutively activating CREB signaling; Wu, L., Liu, J., Gao, P., Nakamura, M., Cao, Y., Shen, H., and Griffin, J. D.; Transforming activity of MECT1-MAML2 fusion oncoprotein is mediated by constitutive CREB activation. Embo J, 2005, 24: 2391-2402 and Coxon, A., Rozenblum, E., Park, Y. S., Joshi, N., Tsurutani, J., Dennis, P. A., Kirsch, I. R., and Kaye, F. J.; Mect1-Mam12 fusion oncogene linked to the aberrant activation of cyclic AMP/CREB regulated genes. Cancer Res, 2005, 65: 7137-7144. This report is consistent with a model where the CRTC1 motif re-directs the strong coactivator MAML2 to CREB. It has also been demonstrated that both the CREB-binding domain and the TAD of CRTC1-MAML2 are required for the fusion to induce foci formation in vitro; Wu et al., supra. The direct evidence that CRTC1-MAML2 is an oncogene, coupled with its high frequency in MECs suggests that this CRTC1-MAML2 protein or its downstream targets are of therapeutic interest.

### SUMMARY OF THE INVENTION

The present application is directed to a method for inhibiting growth or proliferation of mucoepidermoid carcinoma cells comprising administering to a patient in need thereof in an amount that is effective to inhibit growth or proliferation of the mucoepidermoid carcinoma cells a compound of the formula (I)

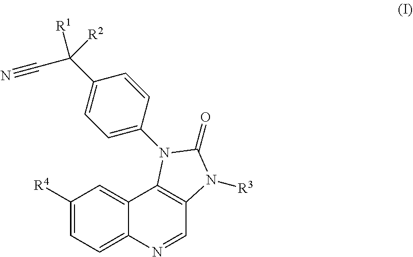

wherein R¹ and R² are each independently methyl or ethyl;
R³ is lower alkyl; and
R⁴ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; or quinoxalinyl; or a pharmaceutically acceptable salt thereof.

The present invention also relates to a method for treating mucoepidermoid carcinoma comprising administering to a patient in need thereof in an amount that is effective to inhibit growth or proliferation of the mucoepidermoid cells a compound of formula (I).

In a further embodiment, the invention relates to a method for inhibiting growth or proliferation of mucoepidermoid carcinoma cells comprising administering to a patient in need thereof in an amount that is effective to inhibit growth or proliferation of the mucoepidermoid cells a compound of formula (I) in combination with a PDE4B inhibitor.

In a still further embodiment, the invention relates to a method for treating mucoepidermoid carcinoma comprising administering to a patient in need thereof in an amount that is effective to inhibit growth or proliferation of the mucoepidermoid cells a compound of formula (I) in combination with a PDE4B inhibitor.

In a further embodiment, the invention relates to a method for inhibiting growth or proliferation of mucoepidermoid carcinoma cells comprising administering to a patient in need thereof in an amount that is effective to inhibit growth or proliferation of the mucoepidermoid cells a PDE4B inhibitor, a PI3-kinase inhibitor or a PDE4B inhibitor and a PI3-kinase inhibitor.

### BRIEF DESCRIPTION OF THE DRAWINGS

FIG. 1 illustrates identification of CRTC1-MAML2 Target genes. A. qPCR analysis showed shRNA #D and #A down-regulated CRTC1-MAML2 expression in H292 and H3118 cells compared to luciferase (sh LUC) control. RT-qPCR experiments were performed in triplicates. Columns: mean, bars: SD (n=3). *p<0.05, compared with expression from shLUC infected cells (Student t test). B Inhibition of CRTC1-MAML2 inhibited H3118 and H292 cell growth. Columns: mean, bars: SD (n=3). *p<0.05, **p<0.01, ***p<0.001, compared with shLUC. C. qPCR analysis showed PDE4B expression was down regulated in H292 and H3118 cells by knock-down of CRTC1-MAML2. *p<0.05, compared with RNA expression from shLUC infected cells. Columns: mean, bars: SD (n=3). D. Western-blot analysis showed the down-regulation of PDE4B protein after CRTC1-MAML2 knock-down. * indicates non-specific band. Image was cropped and auto-leveled by Photoshop. A representative of three independent experiments.

FIG. 2 illustrates inhibition of PDE4B by pharmacological inhibitor rolipram plus forskolin prevents cell growth, causes cell cycle arrest and induces apoptosis in fusion positive MEC cells. Rolipram (R), forskolin (FK), Rolipram plus forskolin (R+FK) or DMSO. Columns: mean, bars: SD (n>3), *p<0.05, **p<0.01, ***p<0.001, compared to HSY cell growth. B. 1×10⁵ cells were treated with 50 μM R plus 20 μM FK or DMSO for 24 hours. C. 1×10⁵ cells were treated with 50 μM rolipram plus 20 μM forskolin with or DMSO for 48 hours. Apoptosis assay was performed by PI and Annexin-V staining and analyzed by flow cytometry. Columns: mean, bars: SD (n=3). *p<0.05, compared with DMSO treated cells.

FIG. 3 illustrates knock-down of PDE4B inhibits fusion positive MEC cell growth, causes cell cycle arrest and induces apoptosis. A. H3118 cell lysate was prepared on day 5 post-transduction and subjected to western blot analysis. Image was cropped and auto-leveled by Photoshop. A representative of three independent experiments. B. 1×10⁵ MEC cells were infected twice by shRNA No. 1 and No. 2 and cell number was count on day 5 post-transduction by trypan blue exclusion. Columns: mean, bars: SD (n>3). *p<0.05, **p<0.01, compared with scramble control virus infected cell growth. C. 1×10⁵ cells were infected twice by shRNA No. 2. Cell cycle was determined by PI staining followed by FACS analysis on day 5 post-transduction, n=3. D. 1×10⁵ cells were infected twice by shRNA No. 2. Apoptosis assay was performed by PI and Annexin-V staining and analyzed by flow cytometry on day 5 post-transduction, Columns: mean, bars: SD (n=3). *p<0.05, compared with control cells.

FIG. 4 illustrates knock-down of PDE4B inhibited MEC cell growth in vivo. A. Tumor volume in SCID hairless mice. Points: mean of 5 tumors, bars: SD (n=5). ***p<0.001, compared to control tumor. B. Representative picture of the tumors dissected from the mice after sacrifice on day 30. Five pair (shPDE4B vs. scramble control) of tumors was shown (n=5). C. Tumor weight from SCID hairless mice. Tumors were weighted on day 30 at sacrifice. Columns: mean, bars: SD (n=5). ***p<0.001, compared to control tumor weight. D. RT-qPCR analysis of PDE4B expression from the tumor biopsies. Columns: mean, bars: SD (n=3). ***p<0.001, compared to control tumor.

FIG. 5 illustrates inhibition of PDE4B increased cellular cAMP levels and inhibited PI3-kinase signaling. A. Pharmacological inhibitor rolipram plus forskolin treatment increased cAMP levels in MEC cells. Columns: mean, bars: SD (n=3). B. Knock-down of PDE4B increased MEC cell cAMP level. n=3. **p<0.01, compared to control cells. Columns: mean, bars: SD (n=3). C. Western blot analysis showed the phospho-AKT levels after 30 min of 50 μM rolipram plus 20 μM forskolin treatment in H3118 cells. The same blot was stripped and probed with AKT or β-actin antibody. Image was cropped and auto-leveled by Photoshop. A representative of three independent experiments. D. Western blot analysis showed the phospho-AKT levels after PDE4B knock-down.

FIG. 6 illustrates PI3-kinase signaling contributes to MEC cell growth. A. Effects of PI3-kinase inhibitor LY-294002 or Compound A on MEC cell growth. (Left) Western blot analysis showed phospho-AKT levels in H3118 cells after 1 hour of inhibitor treatment. The same blot was stripped and probed with anti-AKT or anti-V.-actin antibody. Image was cropped and auto-leveled by Photoshop. A representative of three independent experiments. (Right) MEC cell growth was measured by MTS assay on day 5 of LY-294002 or Compound A treatment. Points: mean, bars: SD (n=3). B. Effects of knock-down of PI3-kinase α or β on MEC cell growth. (Upper) Western blot analysis showed PI3-kinase α or β protein levels after lenti-viral infection. H3118 cell lysates were prepared on day 5 post-transduction. Image was cropped and auto-leveled by Photoshop. (Lower) Effects of knock-down of PI3-kinase α or β on MEC cell growth. 1×10⁵ cells were transduced twice with lenti-viruses harboring shRNAs targeting PI3-K α, β or scramble control. Cell numbers were counted by trypan blue exclusion on day 5 posttransduction. Cell growth of scramble control virus infected cells was set to 100%, Columns: mean, bars: SD (n=3). **p<0.01, ***p<0.001 compared to control cells. C. Effects of Compound A in combination of rolipram plus forskolin on of MEC cell growth. 1×10⁵ cells were treated with the combination of inhibitors listed. Cell numbers were counted by trypan blue exclusion on day 5, Columns: mean, bars: SD (n=3).

FIG. 7 illustrates qPCR analysis showing the down-regulation of MAML2 expression in MEC cells infected with retro-viruses harboring shRNA #A and #D. Total RNA was collected 48 hours post-transduction and the experiments were performed in triplicates. Columns: mean, bars: SD (n=3). *p<0.05, **p<0.01 compared with shLUC treated cells.

FIG. 8 illustrates qPCR validation of the expression changes of Notch target gene HES2 and known CREB target genes in H3118 cells from array analysis. Columns: mean, bars: SD (n=3).

FIG. 9 is a table illustrating genes regulated by CRTC1-MAML2 knockdown in H3118 cells (cut off fold>3 or <−3, p<0.05) using Affymetrix U133A plus_{—}2 microarrays.

### DETAILED DESCRIPTION OF THE INVENTION

The current invention relates to the discovery that imidazoquinolines, as set forth in formula (I), are useful for inhibiting growth or proliferation of mucoepidermoid carcinoma cells. The therapeutic and prophylactic treatments provided by this invention are practiced by administering to a patient in need thereof an amount of a compound of formula (I) that is effective to inhibit growth or proliferation of the mucoepidermoid carcinoma cells or for the treatment of mucoepidermoid carcinoma. In additional embodiments, the imidazoquinolines are administered in combination with a PDE4B inhibitor. In additional embodiments, a PDE4B inhibitor is administered in an amount that is effective to inhibit growth or proliferation of the mucoepidermoid carcinoma cells or for the treatment of mucoepidermoid carcinoma.

The general terms used hereinbefore and hereinafter preferably have within the context of this disclosure the following meanings, unless otherwise indicated:

The prefix “lower” denotes a radical having up to and including a maximum of 7 carbon atoms, preferably from 1 to 4 carbon atoms, the radicals in question being either linear or branched.

Where the plural form is used for compounds, salts, and the like, this is taken to mean also a single compound, salt, or the like.

In a preferred embodiment, “alkyl” has up to a maximum of 12 carbon atoms and is especially lower alkyl.

“Lower alkyl” is preferably “alkyl” with from and including 1 to and including 7 carbon atoms, preferably from 1 to 4 carbon atoms, and is linear or branched; preferably, lower alkyl is butyl, such as n-butyl, sec-butyl, isobutyl, tert-butyl, propyl, such as n-propyl or isopropyl, ethyl or preferably methyl.

“Cycloalkyl” is preferably cycloalkyl with from and including 3 up to and including 6 carbon atoms in the ring; cycloalkyl is preferably cyclopropyl, cyclobutyl, cyclopently or cyclohexyl.

The term “halogen” refers to fluorine, chlorine, bromine, and iodine.

“Alkyl” which is substituted by halogen is preferably perfluoro alkyl such as trifluoromethyl.

As used herein, the term “inhibit”, “inhibiting”, or “inhibit the growth or proliferation” of the mucoepidermoid carcinoma cell refers to slowing, interrupting, arresting or stopping the growth of the mucoepidermoid cell, and does not necessarily indicate a total elimination of the mucoepidermoid carcinoma cell growth. The terms “inhibit” and “inhibiting”, or the like, denote quantitative differences between two states, refer to at least statistically significant differences between the two states. For example, “an amount effective to inhibit growth of mucoepidermoid carcinoma cells” means that the rate of growth of the cells will be at least statistically significantly different from the untreated cells. Such terms are applied herein to, for example, rates of cell proliferation

“Treating”, “treat”, or “treatment” within the context of the instant invention, means an alleviation of symptoms associated with a disorder or disease, or halt of further progression or worsening of those symptoms, or prevention or prophylaxis of the disease or disorder. For example, within the context of this invention, successful treatment may include an alleviation of symptoms related to mucoepidermoid carcinoma or a halting in the progression of a disease such as PHTS.

“Mucoepidermoid carcinoma” refers to a distinct type of tumor containing three cellular elements in varying proportions: squamous cells, mucus-secreting cells, and intermediate cells. Mucoepidermoid carcinomas are the most common malignant salivary gland tumors and the second most frequent lung tumors of bronchial gland origin.

As used herein, the term “pharmaceutically acceptable salts” include those salts formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, malonic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 4-toluenesulfonic acid, 2-naphthalenesulfonic acid, 1,5-naphthalene-disulfonic acid, 2- or 3-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

The term “PDE4B inhibitor” refers to any compound capable of inhibiting the expression or activity of PDE4B, that is to say, in particular, any compound inhibiting the transcription of the gene, the maturation of RNA, the translation of mRNA, the posttranslational modification of the protein, the enzymatic activity of the protein, the interaction of same with a substrate, etc. In some embodiments, the PDE4B inhibitor may be a short hairpin RNA (shRNA) sequence. The term “short hairpin RNA” or “shRNA” refers to RNA molecules having an RNA sequence that makes a tight hairpin turn that can be used to silence gene expression via RNA interference. The shRNA hairpin structure is cleaved by the cellular machinery into siRNA, which is then bound to the RNA-induced silencing complex (RISC). This complex binds to and cleaves mRNAs which match the siRNA that is bound to it. The sequence of the siRNA can correspond to the full length target gene, or a subsequence thereof siRNA is “targeted” to a gene in that the nucleotide sequence of the duplex portion of the siRNA is substantially complementary to a nucleotide sequence of the targeted gene. The siRNA sequence duplex needs to be of sufficient length to bring the siRNA and target RNA together through complementary base-pairing interactions. The siRNA of the invention may be of varying lengths. The length of the siRNA is preferably greater than or equal to ten nucleotides and of sufficient length to stably interact with the target RNA; specifically 10-30 nucleotides; more specifically any integer between 10 and 30 nucleotides, such as 10, 11, 12, 13, 14, 15, 16, 17, 18, 19, 20, 21, 22, 23, 24, 25, 26, 27, 28, 29, and 30. By “sufficient length” is meant a nucleotide of greater than or equal to 10 nucleotides that is of a length great enough to provide the intended function under the expected condition. The shRNA may be cloned into a vector using recombinant DNA techniques.

The terms “substantially identical” or “substantial identity,” in the context of two or more nucleic acids or polypeptide sequences, refer to two or more sequences or subsequences that are the same or have a specified percentage of amino acid residues or nucleotides that are the same (i.e., at least about 60%, preferably 65%, 70%, 75%, preferably 80%, 85%, 90%, 91%, 92%, 93%, 94%, 95%, 96%, 97%, 98% or 99% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence, such as an RNA nucleotide complementary to a DNA nucleotide. Preferably, the substantial identity exists over a region that is at least about 6-7 amino acids or 25 nucleotides in length.

An example of an algorithm that is suitable for determining percent sequence identity and sequence similarity is the BLAST algorithm, which is described in Altschul et al., 1977, Nuc. Acids Res. 25:3389-3402. BLAST is used, with the parameters described herein, to determine percent sequence identity for the nucleic acids and proteins of the invention. Software for performing BLAST analysis is publicly available through the National Center for Biotechnology Information (http://www.ncbi.nlm.nih gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

In a particular embodiment, the compound is an antisense nucleic acid, capable of inhibiting the transcription of the PDE4B or AKAP1 or GABA(A)RAPL1 gene or the translation of the corresponding messenger. The antisense nucleic acid can comprise all or part of the sequence of the PDE4B or AKAP1 or GABA(A)RAPL1 gene, a fragment thereof, the PDE4B or AKAP1 or GABA(A)RAPL1 messenger, or a sequence complementary to same. In particular, the antisense molecule can comprise a region complementary to the sequence comprised between residues 218-2383 of Genbank sequence No. AF208023 or 766-2460 of Genbank sequence No. NM.sub.-002600, and inhibit (or reduce) the translation thereof into protein. The antisense molecule can be a DNA, RNA, ribozyme, etc. It can be single stranded or double stranded. It can also be an RNA coded by an antisense gene. It being an antisense oligonucleotide, it typically comprises fewer than 100 bases, for example approximately 10 to 50 bases. Said oligonucleotide can be modified to improve its stability, its resistance to nucleases, its penetration into the cell, etc.

In some embodiments, practice of the present invention will employ, unless otherwise indicated, conventional techniques of molecular biology, immunology, microbiology, cell biology and recombinant DNA, which are within the skill of the art. See e.g., Sambrook, Fritsch and Maniatis, MOLECULAR CLONING: A LABORATORY MANUAL, (Current Edition); CURRENT PROTOCOLS IN MOLECULAR BIOLOGY (F. M. Ausubel et al. eds., (Current Edition)); the series METHODS IN ENZYMOLOGY (Academic Press, Inc.): PCR 2: A PRACTICAL APPROACH (Current Edition) ANTIBODIES, A LABORATORY MANUAL and ANIMAL CELL CULTURE (R. I. Freshney, ed. (1987)). DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); Fundamental Virology, 2nd Edition, vol. I & II (B. N. Fields and D. M. Knipe, eds.)

According to another embodiment, the compound is a peptide, for example comprising a region of the PDE4B protein and capable of antagonizing the activity of same.

According to another embodiment, the compound is a chemical compound, natural or synthetic, in particular an organic or inorganic molecule of plant, bacterial, viral, animal, eukaryotic, synthetic or semisynthetic origin, capable of modulating the expression or activity of PDE4B. Examples of chemical compounds which are PDE4B inhibitors include rolipram, etazolate, cilomilast, and piclamilast.

Additional examples of PDE4 inhibitors include the following:

Arofylline; Cilomilast (Ariflo, SB-207,499) (a second-generation PDE4 inhibitor with antiinflammatory effects that target bronchoconstriction, mucus hypersecretion, and airway remodeling associated with chronic obstructive pulmonary disease (COPD) (Christensen, Siegfried B.; Guider, Aimee; Forster, Cornelia J.; Gleason, John G.; Bender, Paul E.; Karpinski, Joseph M.; Dewolf, Walter E.; Barnette, Mary S. et al. (1998) “1,4-Cyclohexanecarboxylates: Potent and Selective Inhibitors of Phosophodiesterase 4 for the Treatment of Asthma” Journal of Medicinal Chemistry 41 (6): 821); CP-80633; Denbutylline; Drotaverine (INN, also known as drotaverin) (an antispasmodic drug, structurally related to the opium alkaloid papaverine); Etazolate (SQ-20,009, EHT-0202) (anxiolytic drug; pyrazolopyridine derivative (Hall, Judith A.; Morton, Ian (1999) Concise dictionary of pharmacological agents: properties and synonyms. Kluwer Academic. ISBN 0-7514-0499-3; Williams M (May 1983) “Anxioselective anxiolytics”. Journal of Medicinal Chemistry 26 (5): 619-28. doi:10.1021/jm00359a001. PMID 6132997; Williams M, Risley E A (February 1979) “Enhancement of the binding of 3H-diazepam to rat brain membranes in vitro by SQ 20009, A novel anxiolytic, gamma-aminobutyric acid (GABA) and muscimol”. Life Sciences 24 (9): 833-41. doi:10.1016/0024-3205(79)90367-9. PMID 449623). Etazolate acts as a positive allosteric modulator of the GABAA receptor at the barbiturate binding site, as an adenosine antagonist of the A1 and A2 subtypes, and as a phosphodiesterase inhibitor selective for the PDE4 isoform (Chasin M, Harris D N, Phillips M B, Hess S M (September 1972) “1-Ethyl-4-(isopropylidenehydrazino)-1H-pyrazolo-(3,4-b)-pyridine-5-carboxylic acid, ethyl ester, hydrochloride (SQ 20009)—a potent new inhibitor of cyclic 3′,5′-nucleotide phosphodiesterases”. Biochemical Pharmacology 21 (18): 2443-50. doi:10.1016/0006-2952(72)90414-5. PMID 4345859; Wang P, Myers J G, Wu P, Cheewatrakoolpong B, Egan R W, Billah M M (May 1997) “Expression, purification, and characterization of human cAMP-specific phosphodiesterase (PDE4) subtypes A, B, C, and D”. Biochemical and Biophysical Research Communications 234 (2): 320-4. doi:10.1006/bbrc.1997.6636. PMID 9177268; Gresele, Paolo; Gresele, P. (2002) Platelets in thrombotic and non-thrombotic disorders: pathophysiology, pharmacology and therapeutics. Cambridge, UK: Cambridge University Press. ISBN 0-521-80261-X; Filaminast (primary indication is asthma and COPD treatment (Giembycz, M A (2008) “Can the anti-inflammatory potential of PDE4 inhibitors be realized: guarded optimism or wishful thinking?”. British Journal of Pharmacology 155 (3): 288. doi:10.1038/bjp.2008.297. PMC 2567889. PMID 18660832); Glaucine (alkaloid found in several different plant species; has bronchodilator and antiinflammatory effects, acting as a PDE4 inhibitor and calcium channel blocker, and is used medically as an antitussive in some countries (Riihle K H, Criscuolo D, Dieterich H A, Köhler D, Riedel G. Objective evaluation of dextromethorphan and glaucine as antitussive agents. British Journal of Clinical Pharmacology. 1984 May; 17(5):521-4. PMID 6375709); HT-0712 (experimental cognitive enhancing drug (nootropic)); ICI-63197; Ibudilast (current development codes: AV-411 or MN-166) is an antiinflammatory drug used mainly in Japan; neuroprotective and bronchodilator drug used primarily in asthma and stroke therapy. Its inhibitory action is greatest against PDE4, however it also shows significant inhibitor activity against other PDE subtypes. Depending on the dose, it can selectively inhibit PDE4, or it can act as a non-selective phosphodiesterase inhibitor) (Huang Z, Liu S, Zhang L, Salem M, Greig G M, Chan C C, Natsumeda Y, Noguchi K. Preferential inhibition of human phosphodiesterase 4 by ibudilast. Life Sciences. 2006 May 1; 78(23):2663-8.); Irsogladine; Luteolin (flavanoid; peanut extracted supplement; possesses IGF-1 properties as well)(Yu M C, Chen J H, Lai C Y, Han C Y, Ko W C (February 2010) “Luteolin, a non-selective competitive inhibitor of phosphodiesterases 1-5, displaced [3H-rolipram from high-affinity rolipram binding sites and reversed xylazine/ketamine-induced anesthesia”]. Eur. J. Pharmacol. 627 (1-3): 269-75. doi:10.1016/j.ejphar.2009.10.031. PMID 19853596); Mesembrine (alkaloid from herb Sceletium tortuosum); Piclamilast (more potent than Rolipram) (de Visser Y P, Walther F J, Laghmani E H, van Wijngaarden S, Nieuwland K, Wagenaar G T (2008) “Phosphodiesterase-4 inhibition attenuates pulmonary inflammation in neonatal lung injury.” Eur Respir J 31 (3): 633-644); Roflumilast (trade names Daxas, Daliresp)(licensed for treatment of severe chronic obstructive pulmonary disease in the EU by Merck Sharp & Dohme using the tradename Daxas) (http://emc.medicines.org.uk/medicine/23416/SPC/DAXAS; 500 micrograms film-coated tablets); Ro20-1724; Rolipram (used as investigative tool in pharmacological research; is being studied as a possible alternative to current antidepressants (Bobon D, Breulet M, Gerard-Vandenhove M A, Guiot-Goffioul F, Plomteux G, Sastre-y-Hernandez M, Schratzer M, Troisfontaines B, von Frenckell R, Wachtel H. (1988) “Is phosphodiesterase inhibition a new mechanism of antidepressant action? A double blind double-dummy study between rolipram and desipramine in hospitalized major and/or endogenous depressives”. Eur Arch Psychiatry Neurol Sci 238 (1): 2-6. PMID 3063534; Wachtel H. (1983) “Potential antidepressant activity of rolipram and other selective cyclic adenosine 3′,5′-monophosphate phosphodiesterase inhibitors”. Neuropharmacology 22 (3): 267-72. doi:10.1016/0028-3908(83)90239-3. PMID 6302550). Recent studies show that rolipram may have antipsychotic effects (Maxwell C R, Kanes S J, Abel T, Siegel S J. (2004) “Phosphodiesterase inhibitors: a novel mechanism for receptor-independent antipsychotic medications”. Neuroscience 129 (1): 101-7. doi:10.1016/j.neuroscience.2004.07.038. PMID 15489033; Kanes S J, Tokarczyk J, Siegel S J, Bilker W, Abel T, Kelly M P. (2006) “Rolipram: A specific phosphodiesterase 4 inhibitor with potential antipsychotic activity”. Neuroscience 144 (1): 239-46. doi:10.1016/j.neuroscience.2006.09.026. PMID 17081698). Rolipram shows promise in ameliorating Alzheimer's disease (Smith, Donna L; Pozueta J, Gong B, Arancio O, Shelanski M (Sep. 14, 2009) “Reversal of long-term dendritic spine alterations in Alzheimer disease models”. Proceedings of the National Academy of Sciences of the United States 106 (39): 16877-16882. doi:10.1073/pnas.0908706106. PMC 2743726. PMID 19805389), Parkinson's disease (M F, Beal; Cleren C, Calingasan N Y, Yang L, Klivenyi P, Lorenzl S (2005. “Oxidative Damage in Parkinson's Disease”. U.S. Army Medical Research and Materiel CommandFort Detrick, Md. 21702-5012), and in the regeneration of severed spinal cord axonal bodies (Nikulina, E. (Jun. 8, 2004) “The Phosphodiesterase Inhibitor Rolipram Delivered after a Spinal Cord Lesion Promotes Axonal Regeneration and Functional Recovery”. Proceedings of the National Academy of Sciences of the United States 101: 8786. doi:10.1073/pnas.0402595101). Rolipram is under preclinical investigation for treatment of spinal cord injuries; RPL-554 (LS-193,855) (acts as a long-acting inhibitor of the phosphodiesterase enzymes PDE-3 and PDE-4, producing both bronchodilator and antiinflammatory effects (Boswell-Smith V, Spina D, Oxford A W, Comer M B, Seeds E A, Page C P. The Pharmacology of Two Novel Long-Acting Phosphodiesterase 3/4 Inhibitors, RPL554 (9,10-Dimethoxy-2-(2,4,6-trimethylphenylimino)-3-(N-carbamoyl-2-aminoethyl)-3,4,6,7-tetrahydro-2H-pyrimido(6,1-a)isoquinolin-4-one) and RPL565 (6,7-Dihydro-2-(2,6-diisopropylphenoxy)-9,10-dimethoxy-4H-pyrimido(6,1-a)isoquinolin-4-one). Journal of Pharmacology and Experimental Therapeutics 2006; 318(2):840-848).; YM-976

Examples of PDE4B inhibitors tested in clinical trials or currently in clinical trials include the following:

Cilomilast (Ariflo, SB-207,499) is currently in clinical development by GlaxoSmithKline for COPD treatment; Drotaverine (INN, also known as drotaverin) A few small 2003 studies found drotaverine to be nearly 80% effective in treating renal colic (Romics I, Molnar D L, Timberg G, et al. (July 2003) “The effect of drotaverine hydrochloride in acute colicky pain caused by renal and ureteric stones”. BJU International 92 (1): 92-6. doi:10.1046/j.1464-410X.2003.04262.x. PMID 12823389; Garmish O S, Zabashnyi S I, Smirnova E V, Kobeliatski\check{i} IuIu (February 2003) “[Preparation no-shpa forte for the treatment of renal colic]” (in Russian). Klinichna Khirurhiia (2): 47-50. PMID 12784437) Drotaverine has also been studied in accelerating labor by speeding up cervical dilation, but the results have been conflicting (Singh K C, Jain P, Goel N, Saxena A (January 2004) “Drotaverine hydrochloride for augmentation of labor”. International Journal of Gynaecology and Obstetrics 84 (1): 17-22. doi:10.1016/50020-7292(03)00276-5. PMID 14698825; Madhu C, Mahavarkar S, Bhave S (July 2009) “A randomised controlled study comparing Drotaverine hydrochloride and Valethamate bromide in the augmentation of labour”. Archives of Gynecology and Obstetrics 282 (1): 11-5. doi:10.1007/s00404-009-1188-8. PMID 19644697; Gupta B, Nellore V, Mittal S (March 2008) “Drotaverine hydrochloride versus hyoscine-N-butylbromide in augmentation of labor”. International Journal of Gynaecology and Obstetrics 100 (3): 244-7. doi:10.1016/j.ijgo.2007.08.020. PMID 18031745). Drotaverine has been shown to be effective in paracervical block in managing pain during hysteroscopy and endometrial biopsy when administered together with mefenamic acid (Sharma J B, Aruna J, Kumar P, Roy K K, Malhotra N, Kumar S (June 2009) “Comparison of efficacy of oral drotaverine plus mefenamic acid with paracervical block and with intravenous sedation for pain relief during hysteroscopy and endometrial biopsy”. Indian Journal of Medical Sciences 63 (6): 244-52. doi:10.4103/0019-5359.53394. PMID 19602758). IBS patients presenting with predominant diarrhea are more likely to benefit from Buscopan (Khalif I L, Quigley E M, Makarchuk P A, Golovenko O V, Podmarenkova L F, Dzhanayev Y A (March 2009) “Interactions between symptoms and motor and visceral sensory responses of irritable bowel syndrome patients to spasmolytics (antispasmodics)”. Journal of Gastrointestinal and Liver Diseases 18 (1): 17-22. PMID 19337628). Drotaverin has also been tested in combination with rimantadine for antiviral activity against A and B type influenza (Zhilinskaya I N, Konovalova N I, Kiselev O I, Ashmarin I P (2007) “No-Spa and Remantadin are the novel complex preparations that inhibit effectively reproduction of the avian influenza viruses”. Doklady Biological Sciences 414 (1): 249-50. doi:10.1134/50012496607030234). Drotaverin has an adverse effects frequency of 0.9%, side effects being relatively uncommon (Tar A, Singer J (March 2002) “[Safety profile of NO-SPA]” (in Hungarian). Orvosi Hetilap 143 (11): 559-62. PMID 12583325). Drotaverine in sold under brand name No-Spa (Chinoin Pharmaceutical and Chemical Works, Hungary, a member of the Sanofi-Aventis); Etazolate (SQ-20,009, EHT-0202) for the treatment of Alzheimer's disease.; HT-0712; Roflumilast (trade names Daxas, Daliresp) was found to be effective in clinical trials, however it produced several dose-limiting side effects including nausea, diarrhea and headache. Its development is continuing in an attempt to minimize the incidence of side effects while retaining clinical efficacy (Spina, D (October 2008) “PDE4 inhibitors: current status”. British Journal of Pharmacology 155 (3): 308-15. doi:10.1038/bjp.2008.307. ISSN 1476-5381. PMC 2567892. PMID 18660825) In June 2010, Daxas was approved in the EU for severe COPD associated with chronic bronchitis. In March 2011, Daliresp gained FDA approval in the US for reducing COPD exacerbations; RPL-554 (LS-193,855) (being developed by Verona Pharma as a potential treatment for asthma and hayfever.)

Rolipram, also known as 4-(3-cyclopentyloxy-4-methoxyphenyl)pyrrolidin-2-one, has the structure:

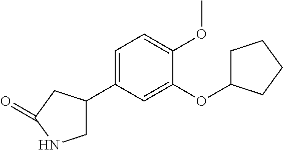 [SMILES: COc1ccc(C2CNC(=O)C2)cc1OC1CCCC1]

Synthesis of rolipram is described in the two citations below. Rolipram is commercially available (i.e. from Cayman Chemical, Sigma-Aldrich). The clinical use of rolipram is limited because of its behavioral and other side effects, and clinical development of rolipram was abandoned due to the side effects associated with its dosing. Newly developed selective PDE IV inhibitors with presumably higher potency and lower toxicity are currently under investigation. Rolipram has been described in Demnitz, J.; LaVecchia, L.; Bacher, E.; Keller, T. H.; Müller, T.; Schu\ddot{r}ch, F.; Weber, H.-P.; Pombo-Villar, E. Enantiodivergent Synthesis of (R)- and (S)-Rolipram. Molecules 1998, 3, 107-119. Rolipram has been described for use in treatment of Alzheimer's Disease in WO 2006/110588. Synthesis of Rolipram has been described in Meng-Yang Chang, Pei-Pei Sun, Shui-Tein Chen, Nein-Chen Chang ChemInform, Volume 35, Issue 19, May 11, 2004.

Compound A is a compound of formula (I), having the chemical name 2-methyl-2-[4-(3-methyl-2-oxo-8-quinolin-3-yl-2,3-dihydroimidazo[4,5-c]quinolin-1-yl)-phenyl]propionitrile, and the structure:

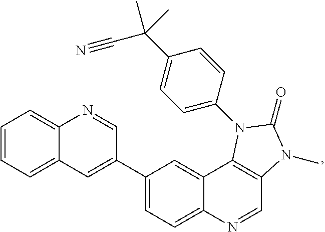 [SMILES: Cn1c(=O)n(-c2ccc(C(C)(C)C#N)cc2)c2c3cc(-c4cnc5ccccc5c4)ccc3ncc21]

or a pharmaceutically acceptable salt thereof. Compound A and a method for preparing this compound is disclosed in International Patent Application WO2006/122806 in Example 7 therein, which is hereby incorporated by reference.

The compounds of formula (I) may be used alone or in compositions together with a pharmaceutically acceptable carrier or excipient. Pharmaceutical compositions of the present invention comprise a therapeutically effective amount of a compound of formula (I) formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The compounds of formula (I) may be administered to humans and other animals orally, parenterally, sublingually, by aerosolization or inhalation spray, rectally, intracisternally, intravaginally, intraperitoneally, bucally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles.

Aerosolized formulations of the invention may be delivered using an aerosol forming device, such as a jet, vibrating porous plate or ultrasonic nebulizer, preferably selected to allow the formation of an aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1-5 μ [symbol] . Predominantly in this application means that at least 70% but preferably more than 90% of all generated aerosol particles are within 1-5 μm rang [symbol] . A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic-Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

A compound of formula (I) can be administered alone or in combination with a PDE4B inhibitor, possible combination therapy taking the form of fixed combinations or the administration of a compound of formula (I) and a PDE4B inhibitor being staggered or given independently of one another. Long-term therapy is equally possible as is adjuvant therapy in the context of other treatment strategies, as described above. Other possible treatments are therapy to maintain the patient's status after tumor regression, or even chemopreventive therapy, for example in patients at risk.

Effective amounts of the compounds of the invention generally include any amount sufficient to detectably inhibit the growth or proliferation of mucoepidermoid carcinoma cells, or by detecting an inhibition or alleviation of symptoms of mucoepidermoid carcinoma. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

According to the methods of treatment of the present invention, mucoepidermoid tumor growth is reduced or prevented in a patient such as a human or lower mammal by administering to the patient an amount of a compound of formula (I), in such amounts and for such time as is necessary to achieve the desired result. An “amount that is effective to inhibit growth or proliferation of the mucoepidermoid carcinoma cells” of a compound of formula (I) refers to a sufficient amount of the compound to treat mucoepidermoid tumor growth, at a reasonable benefit/risk ratio applicable to any medical treatment.

If the compound of formula (I) is administered in combination with a PDE4B inhibitor, the term “amount that is effective to inhibit growth or proliferation of the mucoepidermoid carcinoma cells” is understood to mean that amount of a compound of formula (I) in combination with a specific PDE4B inhibitor to achieve the desired effect. In other words, a suitable combination therapy according to the current invention encompasses an amount of the compound of formula (I) and an amount of PDE4B inhibitor, either of which when given alone at that particular dose would not constitute an effective amount, but administered in combination would be an “amount that is effective to inhibit growth or proliferation of the mucoepidermoid carcinoma cells”.

It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The dose of a compound of the formula (I) or a pharmaceutically acceptable salt thereof to be administered to warm-blooded animals, for example humans of approximately 70 kg body weight, is preferably from approximately 3 mg to approximately 5 g, more preferably from approximately 10 mg to approximately 1.5 g, most preferably from about 100 mg to about 1000 mg per person per day, divided preferably into 1 to 3 single doses which may, for example, be of the same size. Usually, children receive half of the adult dose.

The dose of the PDE4B inhibitor to be administered in combination therapy to warm-blooded animals, for example humans, is preferably from approximately 0.01 mg/kg to approximately 1000 mg/kg, more preferably from approximately 1 mg/kg to approximately 100 mg/kg, per day, divided preferably into 1 to 3 single doses which may, for example, be of the same size. Usually children receive half of the adult dose, and thus the preferential dose range for the PDE4B inhibitor in children is 0.5 mg/kg to approximately 500 mg/kg, per day, divided preferably into 1 to 3 single doses that may be of the same size.

Alternate embodiments of the compounds of formula (I) are given below:

1) Compounds where R¹ is methyl;

2) Compounds where R² is methyl;

3) Compounds where R³ is methyl;

4) Compounds where R⁴ is:
a. Pyridyl unsubstituted or substituted by halogen, cyano, lower alkyl, lower alkoxy or piperazinyl unsubstituted or substituted by lower alkyl;
b. Pyrimidinyl unsubstituted or substituted by lower alkoxy;
c. Quinolinyl unsubstituted or substituted by halogen;
d. Qunionlinyl; or
e. Quinoxalinyl.

It is understood that additional embodiments of the compounds of formula (I) can be selected by requiring one or more of the embodiments (1) through (4) above of the compounds of formula (I). For example, further alternate embodiments can be obtained by combining (1) and (2); (1) and (3); (2) and (3); (1), (2), and (3); (1) and (4)(a); (1) and (4)(b); (1) and (4)(c); (1) and (4)(d); (1) and (4)(e); (2) and (4)(a); (2) and (4)(b); (2) and (4)(c); (2) and (4)(d); (2) and (4)(e); (3) and (4)(a); (3) and (4)(b); (3) and (4)(c); (3) and (4)(d); (3) and (4)(e); (1), (2), and (4)(a); (1), (2), and (4)(b); (1), (2), and (4)(c); (1), (2), and (4)(d); (1), (2), and (4)(e); (1), (3), and (4)(a); (1), (3), and (4)(b); (1), (3), and (4)(c); (1), (3), and (4)(d); (1), (3), and (4)(e); (2), (3), and (4)(a); (2), (3), and (4)(b); (2), (3), and (4)(c); (2), (3), and (4)(d); (2), (3), and (4)(e); (1), (2), (3), and (4)(a); (1), (2), (3), and (4)(b); (1), (2), (3), and (4)(c); (1), (2), (3), and (4)(d); and (1), (2), (3), and (4)(e).

The compounds of formula (I) may be prepared according to PCT Patent Application Publication Number WO 2006/122806, published Nov. 23, 2006, which is hereby incorporated by reference as if fully set forth.

For example, the compounds of formula (I) may be synthesized by reacting a compound of formula (II)

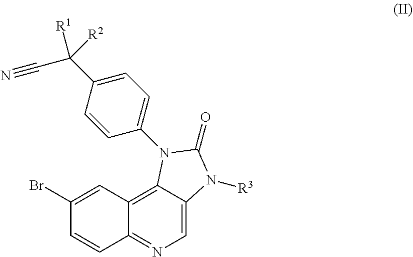

wherein R₁, R₂, and R₃ are as defined for a compound of the formula (I) with a boronic acid of the formula (III)

R₄—B(OH)₂ (III)

or of formula (IIIa)

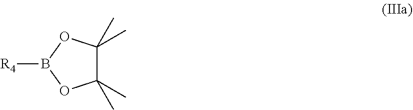

wherein R₄ is as defined for a compound of the formula (I) in the presence of a base and a catalyst in a suitable solvent;
where the above starting compounds (II) and (III) may also be present with functional groups in protected form if necessary and/or in the form of salts, provided a salt-forming group is present and the reaction in salt form is possible;
any protecting groups in a protected derivative of a compound of the formula (I) are removed;
and, if so desired, an obtainable compound of formula (I) is converted into another compound of formula (I), a free compound of formula (I) is converted into a salt, an obtainable salt of a compound of formula (I) is converted into the free compound or another salt, and/or a mixture of isomeric compounds of formula (I) is separated into the individual isomers.

Detailed Description of the Process:

In the more detailed description of the process below, R₁, R₂, R₃, and R₄ are as defined for compounds of formula (I), unless otherwise indicated.

The reaction of compound of formula (II) and (III) is preferably carried out under the conditions of a Suzuki-reaction, preferably in a mixture of a polar aprotic solvent such as DMF and water in the presence of a catalyst, especially a noble metal catalyst, such as palladium (II), preferable bis(triphenylphosphine)palladium (II) dichloride; in the presence of a base such as potassium carbonate.

Protecting Groups

If one or more other functional groups, for example carboxy, hydroxy, amino, or mercapto, are or need to be protected in a compound of formulae (II) or (III), because they should not take part in the reaction, these are such groups as are usually used in the synthesis of peptide compounds, and also of cephalosporins and penicillins, as well as nucleic acid derivatives and sugars.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by acetolysis, protonolysis, solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter.

The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosäuren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide and Derivate” (Chemistry of carbohydrates: monosaccharides and derivatives), Georg Thieme Verlag, Stuttgart 1974.

Additional Process Steps

In the additional process steps, carried out as desired, functional groups of the starting compounds which should not take part in the reaction may be present in unprotected form or may be protected for example by one or more of the protecting groups mentioned hereinabove under “protecting groups”. The protecting groups are then wholly or partly removed according to one of the methods described there.

Salts of a compound of formula I with a salt-forming group may be prepared in a manner known per se. Acid addition salts of compounds of formula (I) may thus be obtained by treatment with an acid or with a suitable anion exchange reagent. A salt with two acid molecules (for example a dihalogenide of a compound of formula (I)) may also be converted into a salt with one acid molecule per compound (for example a monohalogenide); this may be done by heating to a melt, or for example by heating as a solid under a high vacuum at elevated temperature, for example from 130 to 170° C., one molecule of the acid being expelled per molecule of a compound of formula (I).

Salts can usually be converted to free compounds, e.g. by treating with suitable basic compounds, for example with alkali metal carbonates, alkali metal hydrogencarbonates, or alkali metal hydroxides, typically potassium carbonate or sodium hydroxide.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula (I) itself. Enantiomers may be separated through the formation of diastereomeric salts, for example by salt formation with an enantiomer-pure chiral acid, or by means of chromatography, for example by HPLC, using chromatographic substrates with chiral ligands.

It should be emphasized that reactions analogous to the conversions mentioned in this chapter may also take place at the level of appropriate intermediates.

General Process Conditions

All process steps described here can be carried out under known reaction conditions, preferably under those specifically mentioned, in the absence of or usually in the presence of solvents or diluents, preferably such as are inert to the reagents used and able to dissolve these, in the absence or presence of catalysts, condensing agents or neutralizing agents, for example ion exchangers, typically cation exchangers, for example in the H⁺ form, depending on the type of reaction and/or reactants at reduced, normal, or elevated temperature, for example in the range from −100° C. to about 190° C., preferably from about −80° C. to about 150° C., for example at −80 to −60° C., at room temperature, at −20 to 40° C. or at the boiling point of the solvent used, under atmospheric pressure or in a closed vessel, where appropriate under pressure, and/or in an inert atmosphere, for example under argon or nitrogen.

Salts may be present in all starting compounds and transients, if these contain salt-forming groups. Salts may also be present during the reaction of such compounds, provided the reaction is not thereby disturbed.

At all reaction stages, isomeric mixtures that occur can be separated into their individual isomers, e.g. diastereomers or enantiomers, or into any mixtures of isomers, e.g. racemates or diastereomeric mixtures, typically as described under “Additional process steps”.

The solvents from which those can be selected which are suitable for the reaction in question include for example water, esters, typically lower alkyl-lower alkanoates, e.g ethyl acetate, ethers, typically aliphatic ethers, e.g. diethylether, or cyclic ethers, e.g. tetrahydrofuran, liquid aromatic hydrocarbons, typically benzene or toluene, alcohols, typically methanol, ethanol or 1- or 2-propanol, 1-butanol, nitriles, typically acetonitrile, halogenated hydrocarbons, typically dichloromethane, acid amides, typically dimethylformamide, bases, typically heterocyclic nitrogen bases, e.g. pyridine, carboxylic acids, typically lower alkanecarboxylic acids, e.g. acetic acid, carboxylic acid anhydrides, typically lower alkane acid anhydrides, e.g. acetic anhydride, cyclic, linear, or branched hydrocarbons, typically cyclohexane, hexane, or isopentane, or mixtures of these solvents, e.g. aqueous solutions, unless otherwise stated in the description of the process. Such solvent mixtures may also be used in processing, for example through chromatography or distribution.

The compounds of formula (I), including their salts, are also obtainable in the form of hydrates, or their crystals can include for example the solvent used for crystallization (present as solvates).

In the preferred embodiment, a compound of formula (I) is prepared according to or in analogy to the processes and process steps defined in the Examples.

Starting Materials

New starting materials and/or intermediates, as well as processes for the preparation thereof, are further disclosed in International Patent Application No. WO 206/122806.

Starting materials of the formula (II) and (III) are known, commercially available, or can be synthesized in analogy to or according to methods that are known in the art.

For example, a compound of the formula (II) can be prepared by the alkylation of an amino compound of the formula (IV),

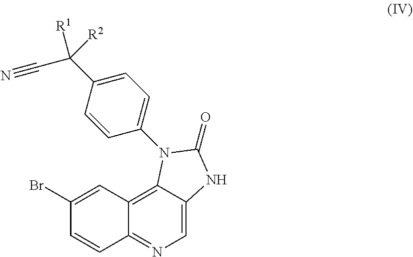

wherein R¹ and R² have the meanings as given under formula (I) with a compound of formula (V)

R³—X (V)

wherein R³ has the meaning as given under formula (I) and X is halogen or another suitable leaving group, in the presence of a base, e.g. sodium hydroxide, in a suitable solvent, e.g. a mixture of dichloromethane and water, preferably in the presence of a phase transfer catalyst, e.g. tetrabutylammonium bromide, at a temperature between 0° C. and 50° C., preferably at room temperature.

A compound of the formula (IV) can be prepared by the cyclization of a diamino compound of the formula (VI),

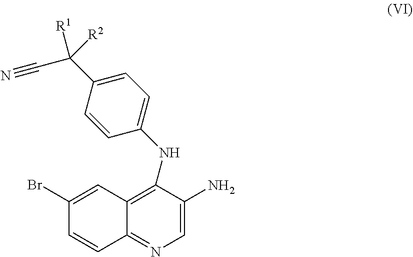

wherein R¹ and R² have the meanings as given under formula (I) with trichloromethyl chloroformate in the presence of a base, such as triethylamine in an appropriate solvent, such as dichloromethane.

A compound of the formula (VI) can be prepared by the reduction of a nitro compound of the formula (VII),

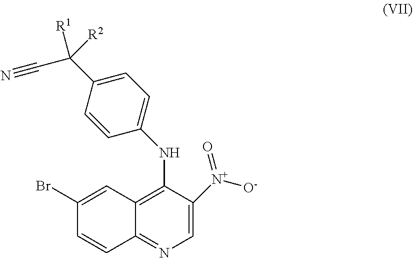

wherein R¹ and R² have the meanings as given under formula (I).

The reduction preferably takes place in the presence of a suitable reducing agent, such as hydrogen in the presence of an appropriate catalyst, such as Raney nickel under pressure, e.g. between 1.1 and 2 bar, in an appropriate solvent, e.g. an alcohol or ether, such as methanol or tetrahydrofurane or a mixture thereof. The reaction temperature is preferably between 0 and 80° C., especially 15 to 30° C.

A compound of the formula (VII) can be prepared by reaction of a compound (VIII)

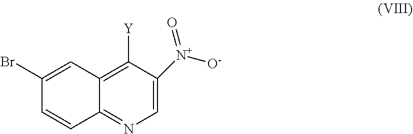

wherein Y is halogen or another suitable leaving group, is reacted with a compound of the formula (IX),

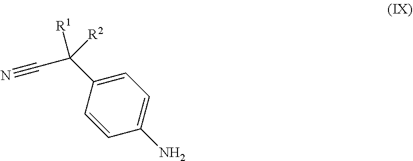

wherein R¹ and R² are as defined for a compound of the formula (I), at a temperature between 0° C. and 50° C., preferably at room temperature in a suitable solvent, i.e. acetic acid.

All remaining starting materials such as starting materials of the formula (III), (IV) and (V) are known, capable of being prepared according to known processes, or commercially obtainable; in particular, they can be prepared using processes as described in International Patent Application No. WO 206/122806. The following Example serves to illustrate the invention without limiting the invention in its scope.

### Example 1

In the present example, expression arrays were used to identify genes whose expression is altered by CRTC1-MAML2, and showed that one of the target genes, PDE4B, is required for fusion-positive MEC cell growth in vitro and in vivo. The results demonstrate that combined inhibition of PDE4B and PI3-kinase signaling is a therapeutic strategy for treating MECs.

Material and Methods

Cell Line Culture:

H292, H3118, HSY (8), 293T and 293FT (Gifts from Dr. William Hahn's lab, Dana-Farber Cancer Institute, Boston) cells were cultured in Dulbecco's modified Eagle's medium (DMEM) (Mediatech, Manassas, Va.), supplemented with 10% heat inactivated fetal bovine serum (Lonza, Basel, Switzerland) and 1% penicillinstreptomycin (Mediatech, Manassas, Va.). All cell lines were incubated at 37° C., 5% CO₂.

Plasmids and Small Hairpin RNAs:

Small hairpin RNA (shRNA) sequences targeting the MAML2 moiety of CRTC1-MAML2 (sh #A, sh #D) or Luciferase (sh LUC) were cloned into pSuperRetro-GFP/Neo Vector (Oligoengine Seattle, Wash.). shRNA lenti-viral constructs targeting PDE4B (#1 TRCN0000048821, #2 TRCN0000048819), PI3-kinase α (#1 TRCN0000196582, #2 TRCN0000196795) and PI3-kinase β (#1 TRCN0000039982, #2 TRCN0000010024) were obtained from Dana-Farber RNAi Screening Facility. pLKO.1-scramble control vector was obtained from Addgene (Addgene, Cambridge, Mass.). pMD2-VSV-G and pCMV_dr8_{—}91 were gifts from Dr. William Hahn. shRNA sequences are provided in Table I.

TABLE I

| | | | | SEQ. |
|---|---|---|---|---|
| | | Hairpin Sequence | | ID. |
| Accession | Symbol | Name | Hairpin Sequence | NO. |
| NM_002600 | PDE4B | #1 | CCGGGCTTGAGTAAATCCTACAGTTCTCGAG | 1 |
| | | TRCN0000048821 | AACTGTAGGATTTACTCAAGCTTTTTG | |
| | | #2 | CCGGGCGCAGAGAGTCATTTCTCTACTCGAG | 2 |
| | | TRCN0000048819 | TAGAGAAATGACTCTCTGCGCTTTTTG | |
| AY040324 | CRTC1- | sh #A | GTAATCAACCTAACACATA | 3 |
| | MAML2 | | | |
| | | sh #D | GACAGAGCCTGGTAATGAT | 4 |
| | | shLUC | CATCACGTACGCGGAATAC | 5 |
| NM_006218 | PIK3CA | #1 | GCCGGCCAGATGTATTGCTTGGTAAACTCGA | 6 |
| | | TRCN0000195203 | GTTTACCAAGCAATACATCTGGTTTTTTG | |
| | | #2 | CCCGGGCATTAGAATTTACAGCAAGACTCG | 7 |
| | | TRCN0000196582 | AGTCTTGCTGTAAATTCTAATGCTTTTTTG | |
| NM_006219 | PIK3CB | #1 | CCGGGCGGGAGAGTAGAATATGTTTCTCGA | 8 |
| | | TRCN0000039982 | GAAACATATTCTACTCTCCCGCTTTTTG | |
| | | #2 | CCGGTATCCTGTAGCGTGGGTAAATCTCGAG | 9 |
| | | TRCN0000010024 | ATTTACCCACGCTACAGGATATTTTT | |

Viral Transduction and Infection:

As described in Chen et al. Genes Cancer, 1:822-835 (2010), retroviruses were produced by transfecting sh #A, sh #D or shLUC vectors together with packing plasmid pMD.MLV and pseudotyped envelope pMD2-VSV-G into 293T cells. To produce lenti-virus, lenti-viral vectors targeting PDE4B, PI3-kinase α, PI3-kinase-β or PLKO.1-scramble control plasmid together with packing plasmid pCMV_dr8_{—}91 and pMD2-VSV-G were transfected into 293FT cells. Virus was collected 48 and 72 hours post-transfection. Target cells were infected twice with virus for 6 hours.

Microarray Experiments:

H3118 or HSY cells were transduced twice with retro-virus harboring shLUC or sh#D. GFP-positive cells were sorted 48 hours post-transduction. Total RNA was extracted using Trizol reagents (Invitrogen) and purified by RNAeasy Mini Column (QIAGEN). Hybridization with Affymetrix U133A plus_{—}2 microarray chips was performed and analyzed at Dana-Farber Cancer Institute Microarray Core. The d-Chip analysis software was used for the analysis; Li, C. and Wong, W. H. Proc. Natl. Acad. Sci. USA (2009) 106: 268-273. Genes with three fold expression changes in H3118 cells were considered significant (n=3, p<=0.05).

Western Blot Analysis:

Fifty micrograms of cell extracts were separated by SDS-polyacrylamide gels using standard methods, for example, Chen et al. Genes Cancer, 1:822-835 (2010) and probed with the indicated antibodies as recommended by the manufacturer. Films were scanned by Scanwizard Pro7 software using ArtixScan 1800f scanner (MicroTek). Images were processed and auto-leveled by Adobe Photoshop software (Adobe, San Jose, Calif.).

Antibodies and Inhibitors:

PDE4B (NB100-2562, Novus Biologicals LLC, Littleton, Colo.), phospho-AKT (#9271, Cell signaling Technology, Danvers, Mass.), AKT (#9272, Cell signaling), PI3-Kinase α (#4249, Cell signaling), PI3-Kinase β (#3011, Cell Signaling), β-actin (Sigma). Forskolin (F 3917, Sigma), rolipram (PD-177, ENZO Life Sciences, Plymouth Meeting, Pa.), LY-294002 (#440202, EMB Biosciences, Gibbstown, N.J.), Compound A (Novartis Institute for Biomedical Research, Boston, Mass.).

Quantitative Real-Time Reverse Transcription PCR (qPCR):

Total RNA was isolated by the Trizol method (Invitrogen). qPCR was performed using ABI PRISM 7500 sequence detector (Applied Biosystems, Foster City, Calif.) by SYBR method (Applied Biosystems). The SYBR method uses SYBR Green I dye, which is a highly specific, double-stranded DNA binding dye, to detect PCR product as it accumulates during PCR cycles. All samples were amplified in triplicates. The relative change of transcript amount was normalized with the GAPDH mRNA expression levels (20). Primer sequences are listed in Table II.

TABLE II

| | | | | SEQ. |
|---|---|---|---|---|
| | | | | ID. |
| Accession | Symbol | Forward primer | Reverse primer | NO. |
| NM_002046 | GAPDH | CAATGACCCCTTCATTGACC | GACAAGCTTCCCGTTCTCAG | 10, |
| | | | | 11 |
| NM_004417 | DUSP1 | TCCTGCCCTTTCTGTACCTG | GGACAATTGGCTGAGACGTT | 12, |
| | | | | 13 |
| NM_002135 | NR4A1 | AAAACGCCAAGTACATCTGCC | GGACAACTTCCTTCACCATGC | 14, |
| | | | | 15 |
| NM_173200 | NR4A3 | CAAGAGACGTCGAAACCGATGT | ACGACCTCTCCTCCCTTTCA | 16, |
| | | | | 17 |
| NM_002600 | PDE4B | TGTTGGAAAATCATCACCTTGCT | CTGAGTGTCTGACGCTGCTTCT | 18, |
| | | | | 19 |
| NM_019089 | HES2 | CATGCTTGCCACCTCTTGCT | CCGTGACTGCTTGAGTTGTAGCT | 20, |
| | | | | 21 |
| AY040324 | MECT1- | TTCGAGGAGGTCATGAAGGA | TTGCTGTTGGCAGGAGATAG | 22, |
| | MAML2 | | | 23 |
| NM_032427 | MAML2 | CTAACCCCTGCTCAAATCCA | GCCTTGACAAATGTCGGTTT | 24, |
| | | | | 25 |

Cell Growth, MTS Assays, Cell Cycle Analysis and Apoptosis Assay:

The Methods were described previously (Chen et al. Genes Cancer, 1:822-835 (2010)). Cell growth: 1-2×10⁵ cells were infected with lenti-virus harboring specific shRNAs. On day 5 post infection, cells were counted by trypan blue exclusion. MTS proliferation assay (Promega, Madison, Wis.): 1-2×10³ cells were treated with LY-294002, Compound A or DMSO in the concentration as indicated in FIG. 6A. MTS activities were measured on day 5. Cell cycle analysis: 1×105 cells were treated with inhibitors or infected with viruses. DNA amount was measured by flow cytometry analysis (BD FACScans). Apoptosis assay: 1×10⁵ cells were treated with inhibitors or infected with virus. Cell viability was measured using the Annexin-V-FLUOS Staining Kit (Roche Diagnostics, Indianapolis, Ind.) by flow cytometry analysis (BD FACScans).

cAMP Measurement:

Cells were trypsinized and washed once with 1×PBS after treatment. The cell pellet was re-suspended in 100 μl of 0.1M HCL. cAMP amount was measure by Direct Cyclic AMP Enzyme Immunoassay Kit (#900-066, Assay Designs, Inc., Ann Arbor, Mich.) according to the manufacturer's protocol. The acetylated version of the kit was used.

In Vivo Murine Xenograft Model:

Mice were maintained and treated in accordance with institutional guidelines of Dana Farber Cancer Institute Animal Resource Facilities. H3118 cells were infected twice with shPDE4B-2 or scramble control virus. On day three post-transduction, 5×10⁶ shPDE4B cells were injected subcutaneously (s.c.) into one flank of a SCID hairless (SHO) mice (Charles River Laboratories International, Inc., Wilmington, Mass.) and 5×106 control cells were injected into the opposite flank (Dubrovska et al., Proc Natl Acad Sci USA, 2009, 106: 268-273; Serra et al. Cancer Res, 2008, 68: 8022-8030). A total of five mice were used. The tumor volume was measured every two days starting from day 10, volume=(length×width2)×(π/6). Mice were sacrificed on day 30. The tumors were excised and the weight was measured.

Statistical Analysis:

The values were shown as the mean±SD. Comparison were performed using student's t test (GraphPad Software, Inc, San Diego, Calif.). Significant p values were shown as p<0.05 (*), p<0.01(**), p<0.001(***).

Results

Identification of CRTC1-MAML2 Target Genes

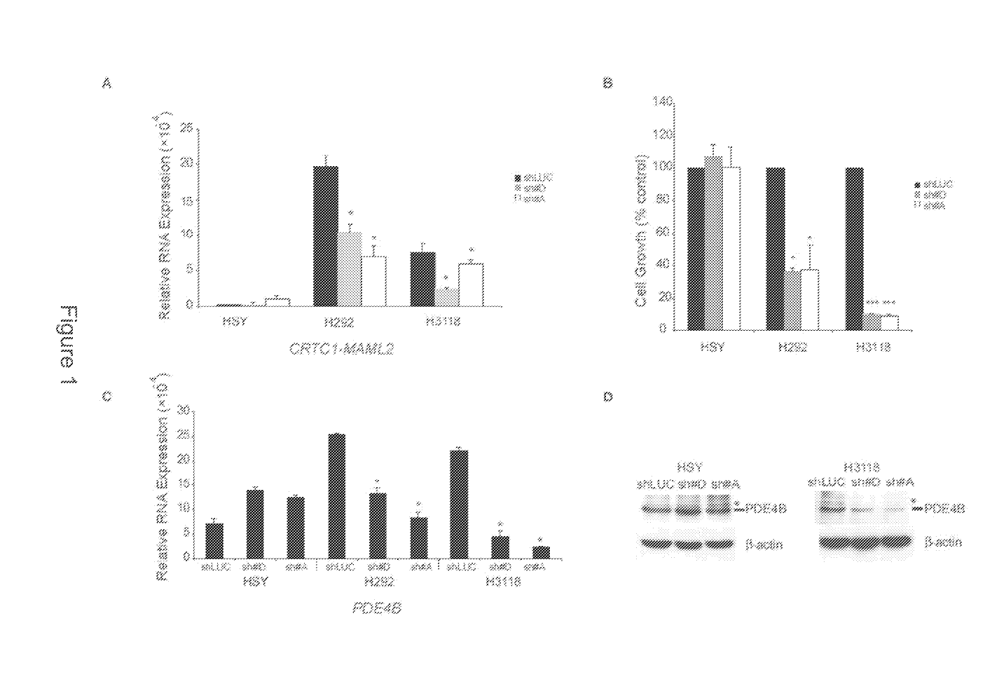

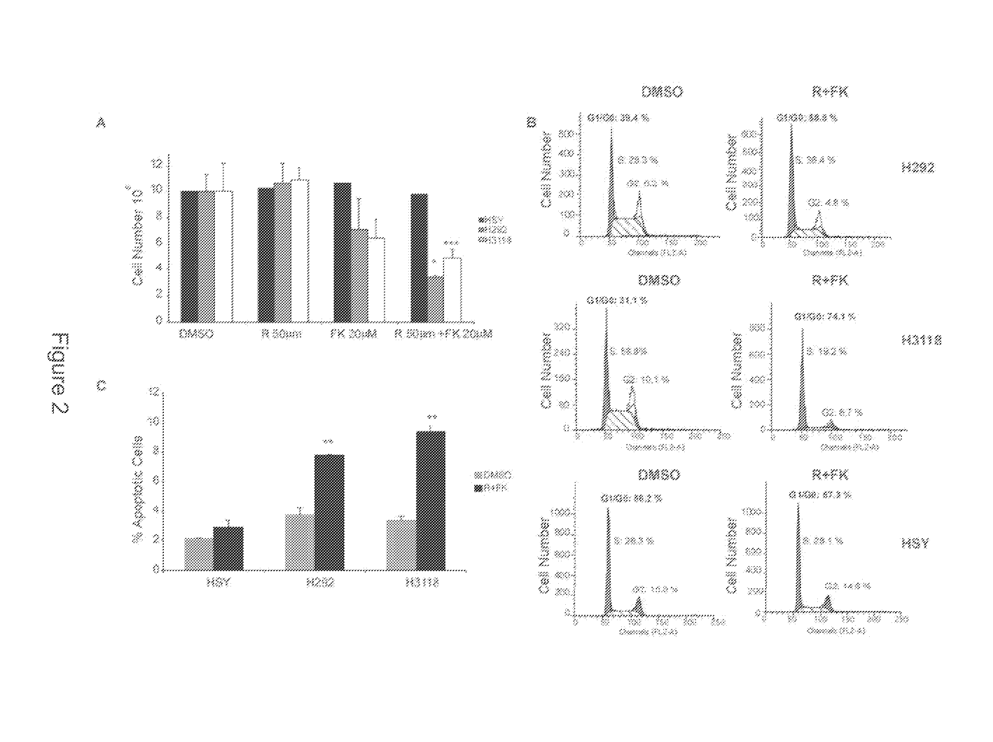

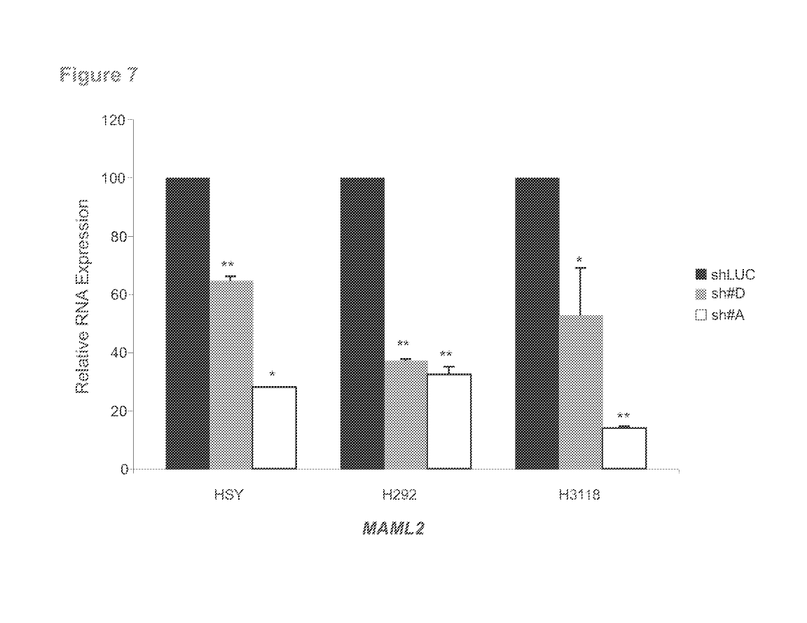

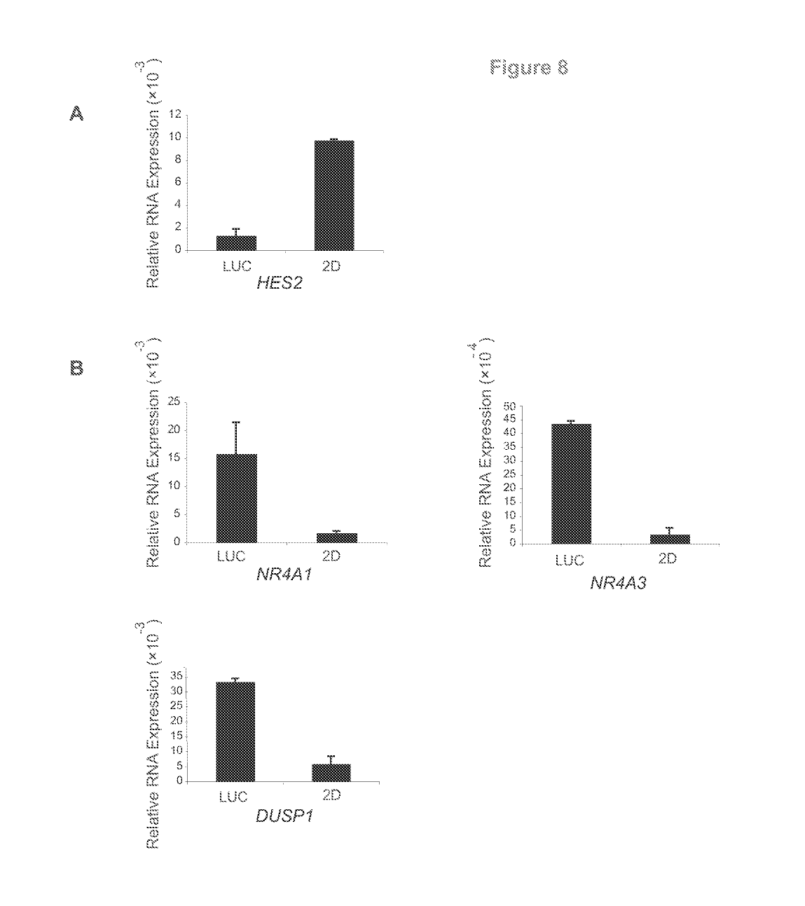

Previous studies aimed at identifying CRTC1-MAML2 target genes (Wu et al., Embo J, 2005, 24: 2391-2402; Coxon et al., Cancer Res, 2005, 65: 7137-7144) were based on over-expressing CRTC1-MAML2 in HeLa cells, which do not carry the fusion gene. Here, a system was employed to identify these target genes by first silencing fusion gene expression via small hairpin RNAs (shRNAs) in fusion-dependent MEC cancer cells, and then examining global gene expression profiles. Two retro-viral constructs were employed carrying a GFP marker and shRNAs (shRNAs #A and #D) targeting the MAML2 moiety of CRTC1-MAML2. Retrovirus carrying a shRNA targeting luciferase (shLUC) was used as a non-silencing control. shRNAs #A and #D specifically inhibited 30%-70% of CRTC1-MAML2 expression in fusion positive MEC H292 (parotid origin) and H3118 cells (pulmonary origin) compared to shLUC. HSY, an immortalized parotid duct cell line was used as the fusion negative control (FIG. 1A). MAML2 was down-regulated to similar extent in all three cell lines (FIG. 7), suggesting effective viral infection. The two forms of shCRTC1-MAML2 (#A and #D) significantly inhibited cell growth in the fusion-positive cells including H292 and H3118 as compared to the shLUC control retrovirus, while having no significant effect on the growth of fusion-negative HSY cells (FIG. 1B). These data indicate that these two forms of shCRTC1-MAML2 used specifically inhibit both the fusion expression and cell growth in the fusion positive MEC cells.

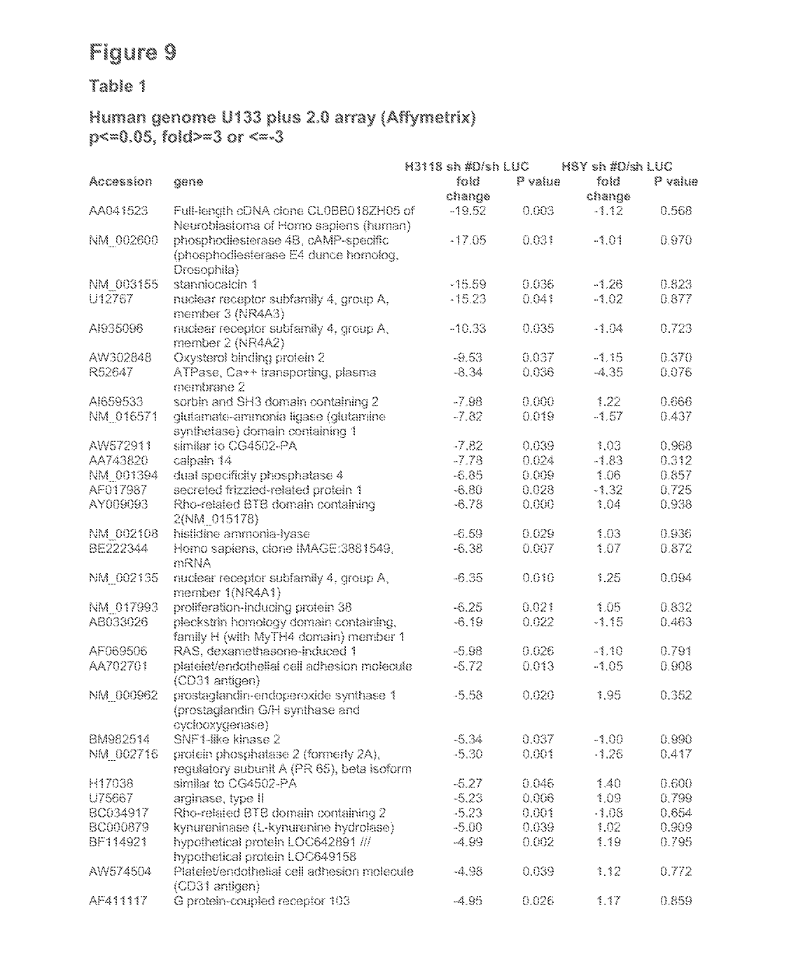

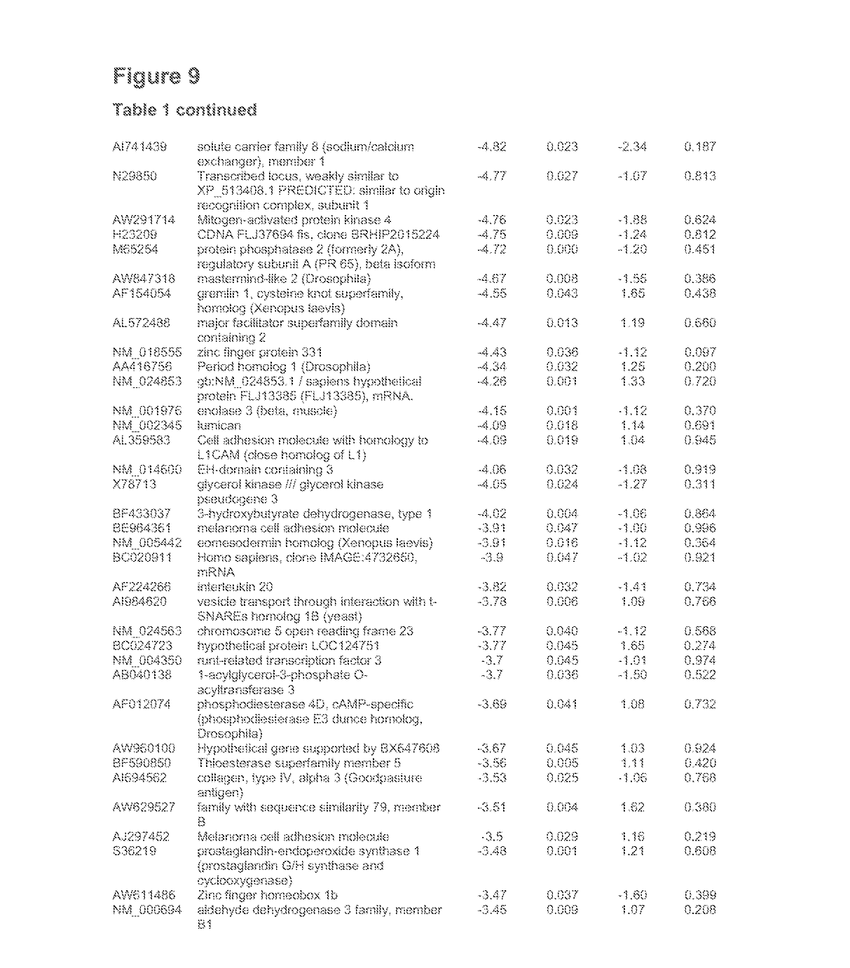

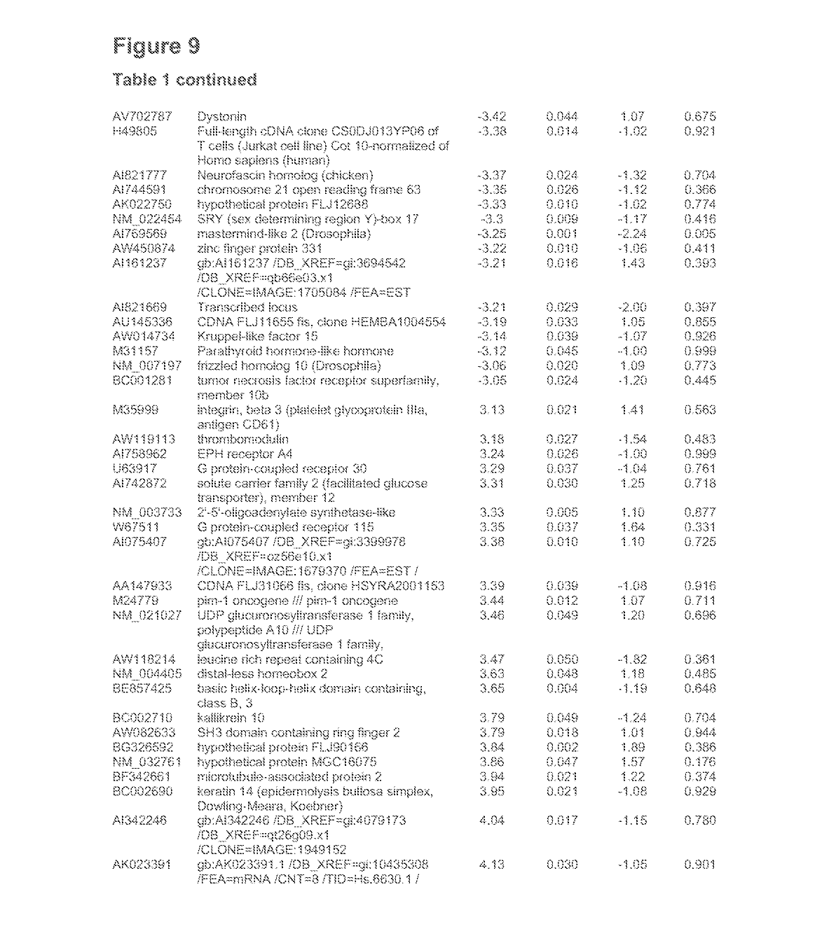

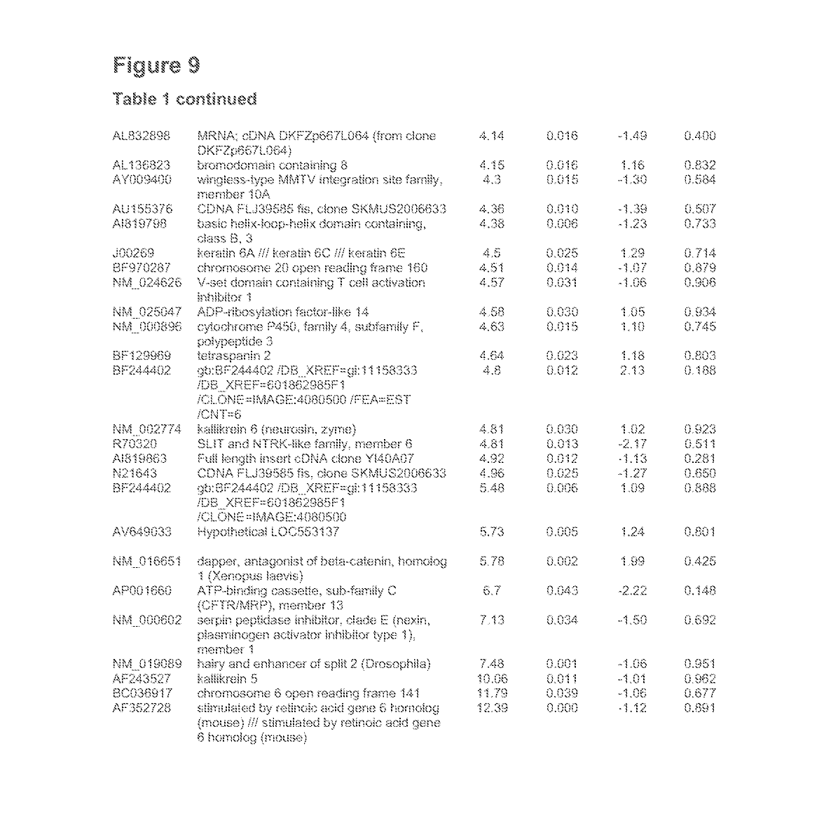

To identify CRTC1-MAML2 target genes, shRNA #D was used to infect H3118 and HSY cells. GFP-positive cells were enriched by flow-cytometry, total RNA was isolated and microarray analysis was performed (U133 plus_{—}2 array, Affimatrix). Expression of 128 genes was found to be altered by greater than or equal to 3 fold in H3118 cells but not in HSY cells, comparing CRTC1-MAML2 knock-down to luciferase knock-down in each cell type (FIG. 9). Among the down-regulated genes were several known CREB target genes, including STC1, NR4A1, NR4A2, NR4A3 and DUSP1 (Tullai et al. J Biol Chem, 2007, 282: 9482-9491; Zhang et al., Proc Natl Acad Sci USA, 2005, 102: 4459-4464). The Notch target gene HES2 was up-regulated 7 fold upon CRTC1-MAML2 knock-down. qPCR assays were conducted to verify the expression changes of selected CREB and Notch target genes (FIG. 8).

In addition to known CREB target genes, we found that expression of phosphodiesterase 4B (PDE4B), was down-regulated 17-fold in H3118 cells upon CRTC1-MAML2 depletion. Further, both shRNAs #A and #D significantly inhibited PDE4B expression in H3118 and H292 cells, but not in fusion negative HSY cells (FIG. 1C). The protein level of PDE4B was reduced by CRTC1-MAML2 depletion in fusion-positive H3118 cells but not in HSY cells (FIG. 1D). PDE4B is a member of the type IV, cyclic AMP (cAMP)-specific phosphodiesterase family that regulates the cellular concentrations of cyclic nucleotides, and is implicated in signal transduction (Houslay, M., Trends Biochem Sci, 35: 91-100, (2010); Lynch et al, Curr Tip Dev Biol, 2006, 75: 225-259). PDE4B has been reported to contribute to the tumor formation in other cancers, such as leukemia and lymphoma (Smith et al. Blood, 2005, 105:308-316). As activation of CREB signaling is one of the mechanisms through which CRTC1-MAML2 exerts its oncogenic activity, the question of whether PDE4B is regulated in a CREB-dependent manner was investigated. The adenylyl cyclase activator forskolin, a cAMP/CREB signaling inducer did not change PDE4B expression in MEC cells (data not shown), suggesting the promoter of PDE4B does not harbor functional CREB sites. This is consistent with the report by Zhang et al. showing that the expression of PED4B was not changed in HEK293T and pancreatic islet cells by forskolin treatment (Zhang et al., Proc Natl Acad Sci USA, 2005, 102: 4459). In addition, there was no detectable CREB and phospho-CBP (CREB Binding Protein) binding on the promoter of PDE4B in HEK293T, hepatocyte and pancreatic islet cells by ChIP on CHIP analysis (Zhang et al., Proc Natl Acad Sci USA, 2005, 102: 4459). Furthermore, the expression of PDE4B was not affected by shRNA-mediated CREB depletion in MEC cells (data not shown). These results suggested that PDE4B might be regulated by CRTC1-MAML2 independent of CREB signaling.

Inhibition of PDE4B by Rolipram, in Combination with Forskolin Treatment Inhibits Cell Growth, Causes Cell Cycle Arrest and Induces Apoptosis in MEC Cells

Besides the functions in regulating cellular cAMP levels, the potential role of PDE4B in CRTC1-MAML2 mediated carcinogenesis in MEC was investigated. Pharmacological inhibitor of PDE4, rolipram together with cAMP activator forskolin inhibited H292 and H3118 cell growth by 50%-60% compared to HSY cells (FIG. 2A). The cell growth was not changed by rolipram treatment alone, but the inhibitory effect was greatly enhanced by combining both rolipram and forskolin, suggesting possible regulation of cAMP signaling in MEC positive cell growth (FIG. 2A). The growth of fusion negative HSY cells was not affected by PDE4B inhibition, indicating that HSY cells do not depend on PDE4B for optimal growth (FIG. 2A), though the expression level of PDE4B is similar in HSY cells compared to H292 and H3118 cells (data not shown). The molecular mechanisms of the cell growth inhibition were further determined by analyzing cell cycle profile and cell apoptotic rate. Twenty four hours of rolipram plus forskolin treatment caused H292 and H3118 cell cycle arrest at G1/G0 phase compared to DMSO control (58% vs. 39% in H292 cells, 74% vs. 31% in H3118 cells). While the cell cycle of HSY was not affected (56% vs. 57%) (FIG. 2C), Rolipram plus forskolin also significantly induced apoptosis in H292 and H3118 cells, but not HSY cells (FIG. 2B). Taken together these data demonstrated that inhibition of PDE4B prevented fusion positive MEC cell growth by inducing cell cycle arrest and apoptosis.

Depletion of PDE4B by shRNA Inhibits Cell Growth, Causes Cell Cycle Arrest and Induces Apoptosis in MEC Cells

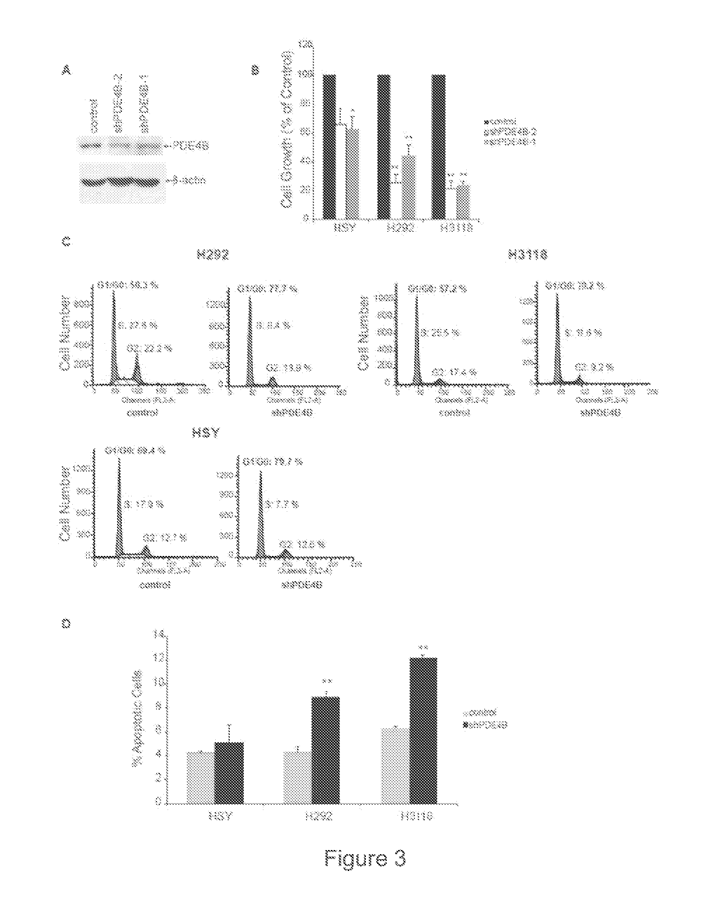

Since more potent and PDE4B isoform-specific inhibitors are still under development (Pages et al., Expert Opin Ther Pat, 2009, 19: 1501-1519; Srivani et al., Curr Pharm Des, 2008, 14: 3854-3872), two shRNAs (#1 and #2) were used to specifically knock-down PDE4B (FIG. 3A). Upon depletion of PDE4B by infecting cells with lenti-viruses harboring the shRNAs, the growth of H292 and H3118 cells was significantly inhibited by 74% and 83% respectively compared to non-specific scramble control (FIG. 3B). The down-regulation of PDE4B caused slight growth reduction in HSY cells, but to a much less extent compared to H292 and H3118 cells. In addition, knock-down of PDE4B caused H292 and H3118 cell cycle arrest at G0/G1 phase compared to scramble control (78% vs. 50% in H292 cells, 79% vs. 57% in H3118 cells), while HSY cells had much less effect (FIG. 3C). Furthermore, H292 and H3118 cells underwent significant apoptosis after PDE4B down-regulation, while HSY cells had no significant effect (FIG. 3D). These data suggested that specific down-regulation of PDE4B by shRNAs inhibited MEC cell growth through cell cycle arrest and apoptosis, which was consistent with the effects of the pharmacological inhibitors of PDE4B on MEC cells.

PDE4B is Required for MEC Cell Growth In Vivo

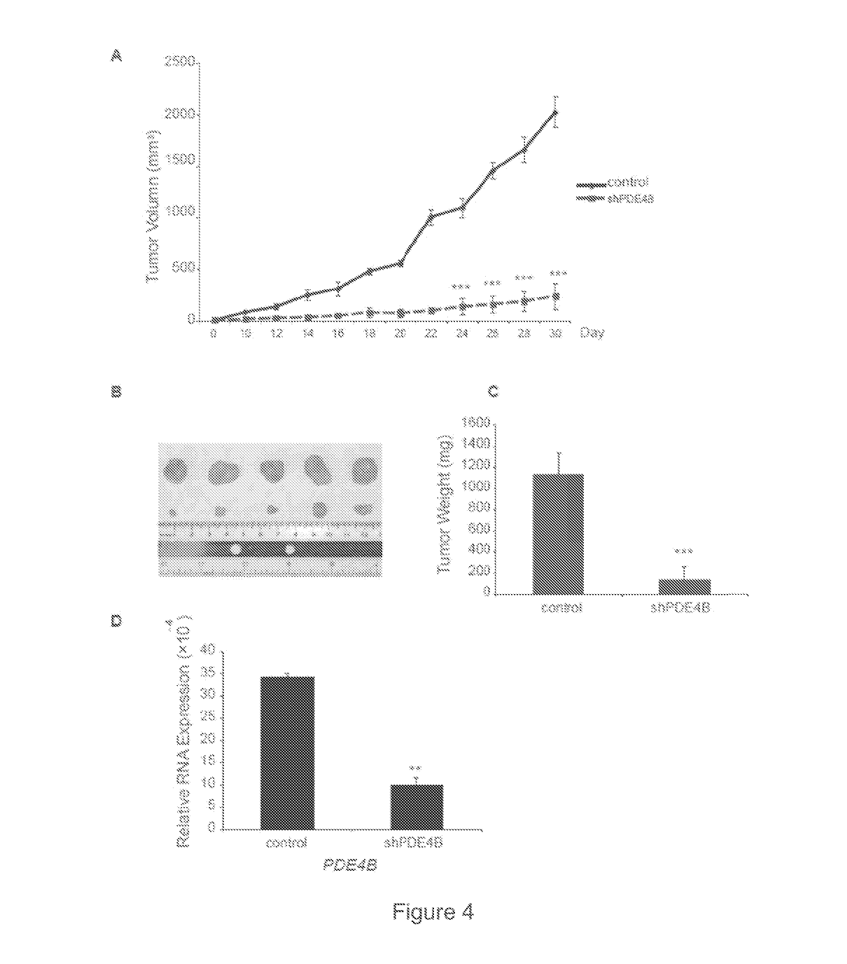

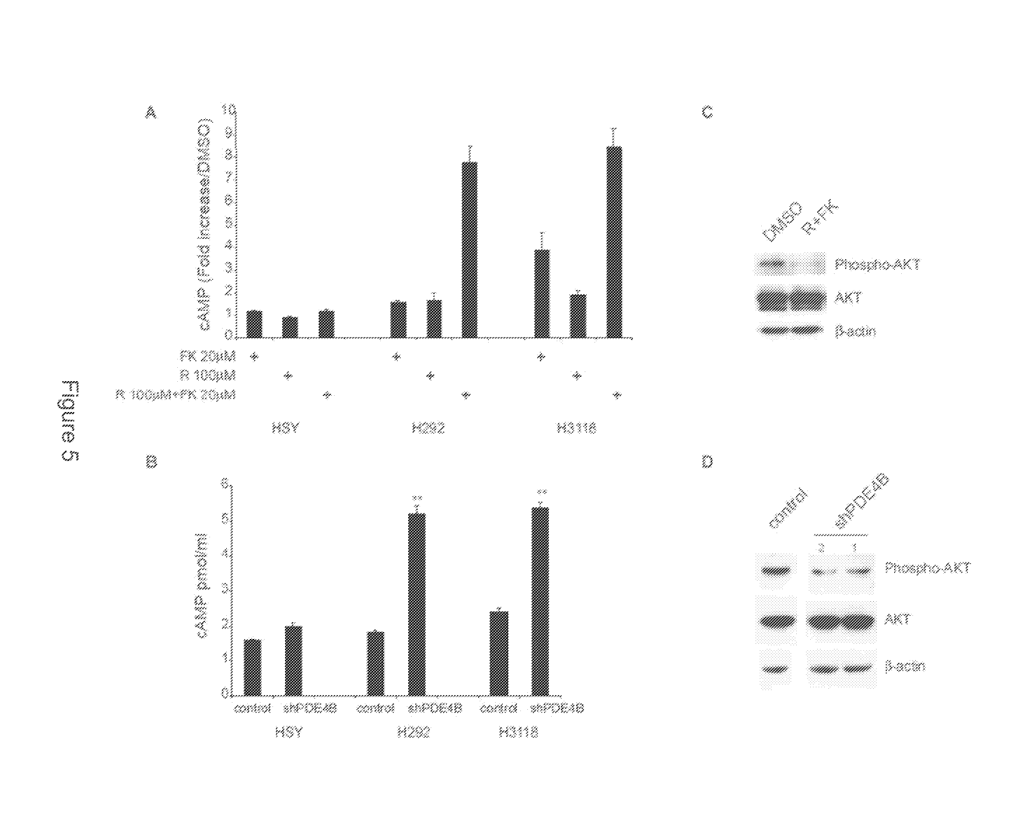

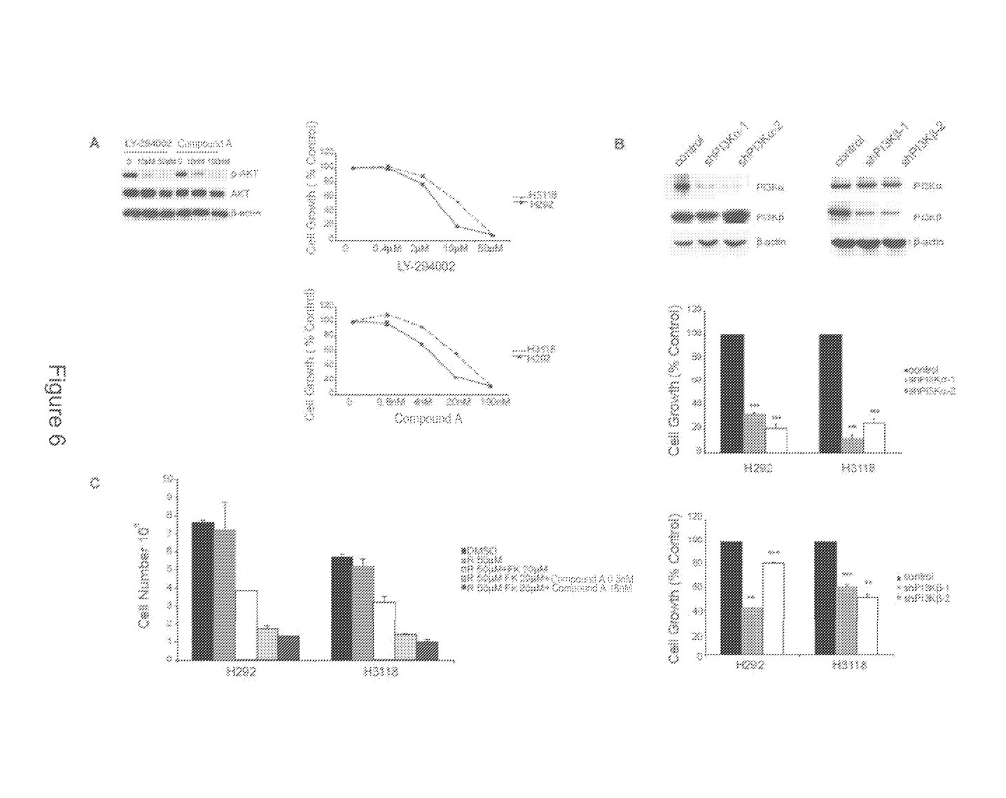

The functional significance of PDE4B in CRTC1-MAML2 mediated carcinogenesis in vivo was also evaluated. Five million H3118 cells infected with lenti-virus harboring shRNA targeting PDE4B (shPDE4B) were injected s.c. into one flank of a SCID hairless mouse and five million control cells were injected into the opposite flank. Tumor volume was measured every two days. Cells with PDE4B knock-down grew significantly slower than control cells in vivo (FIG. 4A). The mice were sacrificed after 30 days and the weights of the tumors were measured. The average weight of the tumors with PDE4B down-regulation was 88% less than the controls (FIG. 4C). The expression of PDE4B was repressed by 71% in the tumor biopsies with PDE4B down-regulation compared to controls (FIG. 4D). Taken together, these data demonstrated that PDE4B has an important role in fusion positive MEC cell growth in vivo.

Inhibition of PDE4B Increases Cellular cAMP Levels in MEC Cells and Down-Regulates PI3-Kinase Signaling

As PDE4B is an enzyme that hydrolyzes cAMP, inhibition of PDE4B was investigated to determine whether inhibition would increase cellular cAMP levels in CRTC1-MAML2 positive cells. As determined, rolipram plus forskolin treatment increased cAMP levels by 7-fold compared to DMSO control in H292 cells. In H3118 cells, forskolin treatment alone increased cAMP levels by 4-fold, while combination of rolipram and forskolin further increased cAMP level to 8-fold. These inhibitors had no effect on HSY cells (FIG. 5A). Specific knockdown of PDE4B by shRNA significantly increased cAMP levels in H292 and H3118 cells compared to scramble control, while HSY cells had little effect (FIG. 5B). These data indicated that PDE4B specifically regulated cAMP levels in fusion positive MEC cells.

As the more potent PDE4B specific inhibitors are yet to be identified, the signaling pathways that are downstream of PDE4B were investigated and more effective inhibitors for the CRTC1-MAML2 signaling were sought. Smith et al. reported that PI3-kinase/AKT signaling is one of the downstream pathways of PDE4B (Smith et al. Blood, 2005, 105:308-316). After inhibiting PDE4B by rolipram plus forskolin or shRNA knock-down, decreased phospho-AKT levels were observed in fusion positive MEC cells compared to control treated cells, indicating that inhibition of PDE4B leads to down-regulation of PI3-kinase/AKT signaling (FIGS. 5C and D).

PI3-Kinase Signaling Contributes to MEC Cell Growth

PI3-kinase/AKT signaling plays an important role in cell growth and survival (Courtney et al., J Clin Oncol, 28: 1075-1083, 2010) and may promote cell growth in a PDE4B-dependent manner in some cancers (Smith et al. Blood, 2005, 105:308-316; McEwan et al., Cancer Res, 2007, 67: 5248-5257). Therefore, the contribution of PI3-kinase signaling to PDE4B mediated tumorigenesis in MEC was investigated. PI3-kinase inhibitors LY-294002 and Compound A inhibited H292 and H3118 cell growth in a dose dependent manner, with IC50 of LY around 4-10 μM and IC50 of Compound A around 6-20 nM (FIG. 6A). MEC cells express PI3-kinase isoforms α, β and δ, with α and β5- to 20-fold more abundant than δ (data not shown). In order to investigate whether specific PI3-kinase isoforms play a more important role in the regulation of MEC cell growth, shRNAs were used to specifically knock-down α and β and investigate the growth inhibitory effects on MEC cells. The down-regulation of PI3-kinase was isoform specific, as knock-down of α had no effects on the protein levels of β and vice versa (FIG. 6B) Inhibition of PI3-kinase α in H292 and H3118 cells inhibited 70%-90% of cell growth, while knock-down of PI3-kinase β reduced 20%-50% of the cell growth (FIG. 6B), suggesting multiple PI3-kinases contribute to MEC cell growth. Moreover, Compound A, a PI3-kinase inhibitor in phase II clinical trials, had additive effect in inhibiting MEC cell growth when combined with rolipram and forskolin (FIG. 6C). These data suggested that PI3-kinase signaling contributed significantly to MEC cell growth, and the combination of PDE4B and PI3-kinase inhibitors could have synergistic activity in MEC.

In summary, PDE4B is a novel CRTC1-MAML2 downstream target gene. Inhibition of PDE4B by either pharmacological inhibitors or shRNAs prevented MEC cell growth in vitro and in vivo through inducing cell cycle arrest and apoptosis. In addition, PI3-kinase signaling significantly contributed to MEC cell growth, demonstrating that the PI3-kinase inhibitors of formula (I), either alone or in combination with PDE4B inhibitors represent a promising therapeutic strategy in treating MECs.

The definitions and disclosures provided herein govern and supersede all others incorporated by reference. Although the invention herein has been described in connection with preferred embodiments thereof, it will be appreciated by those skilled in the art that additions, modifications, substitutions, and deletions not specifically described may be made without departing from the spirit and scope of the invention as defined in the appended claims. It is therefore intended that the foregoing detailed description be regarded as illustrative rather than limiting, and that it be understood that it is the following claims, including all equivalents, that are intended to define the spirit and scope of this invention.

1. A method for inhibiting growth or proliferation of mucoepidermoid carcinoma cells comprising administering to a patient in need thereof in an amount that is effective to inhibit growth or proliferation of the mucoepidermoid carcinoma cells a compound of the formula 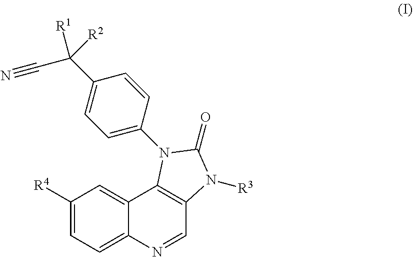
wherein R¹ and R² are each independently methyl or ethyl;
R³ is lower alkyl; and
R⁴ is pyridyl unsubstituted or substituted by halogen, cyano, lower alkyl, lower alkoxy or piperazinyl unsubstituted or substituted by lower alkyl; pyrimidinyl unsubstituted or sub-stituted by lower alkoxy; quinolinyl unsubstituted or substituted by halogen; or quinoxalinyl;
or a pharmaceutically acceptable salt thereof.

2. A method according to claim 1 wherein R¹, R², and R³ are all methyl.

3. A method according to claim 2 wherein R⁴ is pyridyl unsubstituted or substituted by lower alkyl or lower alkoxy.

4. A method according to claim 2 wherein R⁴ is quinolinyl unsubstituted or substituted by halogen.

5. A method according to claim 1 wherein the compound of formula (I) is 2-methyl-2-[4-(3-methyl-2-oxo-8-quinolin-3-yl-2,3-dihydroimidazo[4,5-c]quinolin-1-yl)-phenyl]propionitrile or a pharmaceutically acceptable salt thereof.

6. A method according to claim 1 wherein the amount of the compound or salt of formula (I) that is effective to inhibit growth or proliferation of the mucoepidermoid carcinoma cells is an administered amount ranging from 0.001 to 1000 mg/kg.

7. A method for treating mucoepidermoid carcinoma comprising administering to a patient in need thereof in an amount that is effective to inhibit growth or proliferation of the mucoepidermoid carcinoma cells a compound of the formula 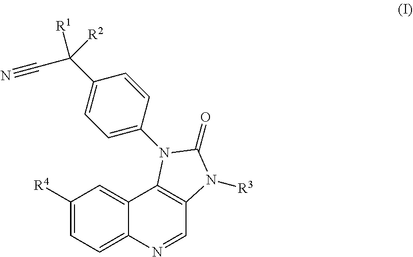
wherein R¹ and R² are each independently methyl or ethyl;
R³ is lower alkyl; and
R⁴ is pyridyl unsubstituted or substituted by halogen, cyano, lower alkyl, lower alkoxy or piperazinyl unsubstituted or substituted by lower alkyl; pyrimidinyl unsubstituted or sub-stituted by lower alkoxy; quinolinyl unsubstituted or substituted by halogen; or quinoxalinyl;
or a pharmaceutically acceptable salt thereof.

8. A method according to claim 7 wherein R¹, R², and R³ are all methyl.

9. A method according to claim 9 wherein R⁴ is pyridyl unsubstituted or substituted by lower alkyl or lower alkoxy.

10. A method according to claim 9 wherein R⁴ is quinolinyl unsubstituted or substituted by halogen.

11. A method according to claim 8 wherein the compound of formula (I) is 2-methyl-2-[4-(3-methyl-2-oxo-8-quinolin-3-yl-2,3-dihydroimidazo[4,5-c]quinolin-1-yl)-phenyl]propionitrile or a pharmaceutically acceptable salt thereof.

12. A method according to claim 8 wherein the amount of the compound or salt of formula (I) that is effective to inhibit growth or proliferation of the mucoepidermoid carcinoma cells is an administered amount ranging from 0.001 to 1000 mg/kg.

13. A method according to claim 1 further comprising administering to the patient
a PDE4B inhibitor.

14. (canceled)

15. (canceled)

16. (canceled)

17. A method according to claim 13 wherein said PDE4B inhibitor is rolipram.

18. A method according to claim 7 further comprising administering to the patient
a PDE4B inhibitor.

19. (canceled)

20. (canceled)

21. (canceled)

22. A method according to claim 18 wherein said PDE4B inhibitor is rolipram.

23. (canceled)

24. (canceled)

25. (canceled)

26. (canceled)

27. (canceled)

28. (canceled)

29. (canceled)

30. (canceled)

31. (canceled)

32. (canceled)

33. A method for inhibiting growth or proliferation of mucoepidermoid carcinoma cells comprising administering to a patient in need thereof in an amount that is effective to inhibit growth or proliferation of the mucoepidermoid carcinoma cells a PDE4B inhibitor, a PI3-kinase inhibitor or a PDE4B inhibitor and a PI3-kinase inhibitor.

34. The method according to claim 33, wherein the PDE4B inhibitor comprises an shRNA, rolipram or forskolin and combinations thereof.

35. The method according to claim 34, wherein the shRNA is selected from the group comprising SEQ ID NO 1, SEQ ID NO 2, SEQ ID NO 3, SEQ ID NO 4 and combinations thereof.

36. The method according to claim 33, further comprising administering to a patient an effective amount of a compound of the formula 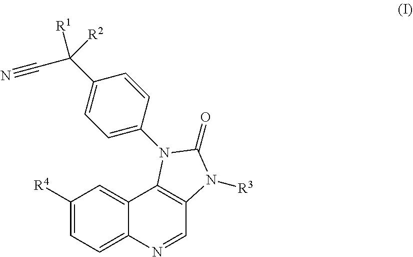
wherein R¹ and R² are each independently methyl or ethyl;
R³ is lower alkyl; and
R⁴ is pyridyl unsubstituted or substituted by halogen, cyano, lower alkyl, lower alkoxy or piperazinyl unsubstituted or substituted by lower alkyl; pyrimidinyl unsubstituted or sub-stituted by lower alkoxy; quinolinyl unsubstituted or substituted by halogen; or quinoxalinyl;
or a pharmaceutically acceptable salt thereof.

37. The method according to claim 35, wherein the PI3-kinase inhibitor comprises a shRNA.

38. The method according to claim 37, wherein the shRNA comprises SEQ ID NO 6, SEQ ID NO 7, SEQ ID NO 8, SEQ ID NO 9, and combinations thereof.


SEQUENCE LISTING (25 sequences)

SEQ ID NO: 1 - DNA, 58 bp, Artificial Sequence
  Synthetic hairpin sequence
ccgggcttgagtaaatcctacagttctcgagaactgtaggatttactcaagctttttg

SEQ ID NO: 2 - DNA, 58 bp, Artificial Sequence
  Synthetic hairpin sequence
ccgggcgcagagagtcatttctctactcgagtagagaaatgactctctgcgctttttg

SEQ ID NO: 3 - DNA, 19 bp, Artificial Sequence
  Synthetic hairpin sequence
gtaatcaacctaacacata

SEQ ID NO: 4 - DNA, 19 bp, Artificial Sequence
  Synthetic hairpin sequence
gacagagcctggtaatgat

SEQ ID NO: 5 - DNA, 19 bp, Artificial Sequence
  Synthetic hairpin sequence
catcacgtacgcggaatac

SEQ ID NO: 6 - DNA, 60 bp, Artificial Sequence
  Synthetic hairpin sequence
gccggccagatgtattgcttggtaaactcgagtttaccaagcaatacatctggttttttg

SEQ ID NO: 7 - DNA, 60 bp, Artificial Sequence
  Synthetic hairpin sequence
cccgggcattagaatttacagcaagactcgagtcttgctgtaaattctaatgcttttttg

SEQ ID NO: 8 - DNA, 58 bp, Artificial Sequence
  Synthetic hairpin sequence
ccgggcgggagagtagaatatgtttctcgagaaacatattctactctcccgctttttg

SEQ ID NO: 9 - DNA, 57 bp, Artificial Sequence
  Synthetic hairpin sequence
ccggtatcctgtagcgtgggtaaatctcgagatttacccacgctacaggatattttt

SEQ ID NO: 10 - DNA, 20 bp, Artificial Sequence
  Synthetic forward primer
caatgaccccttcattgacc

SEQ ID NO: 11 - DNA, 20 bp, Artificial Sequence
  Synthetic reverse primer
gacaagcttcccgttctcag

SEQ ID NO: 12 - DNA, 20 bp, Artificial Sequence
  Synthetic forward primer
tcctgccctttctgtacctg

SEQ ID NO: 13 - DNA, 20 bp, Artificial Sequence
  Synthetic reverse primer
ggacaattggctgagacgtt

SEQ ID NO: 14 - DNA, 21 bp, Artificial Sequence
  Synthetic forward primer
aaaacgccaagtacatctgcc

SEQ ID NO: 15 - DNA, 21 bp, Artificial Sequence
  Synthetic reverse primer
ggacaacttccttcaccatgc

SEQ ID NO: 16 - DNA, 22 bp, Artificial Sequence
  Synthetic forward primer
caagagacgtcgaaaccgatgt

SEQ ID NO: 17 - DNA, 20 bp, Artificial Sequence
  Synthetic reverse primer
acgacctctcctccctttca

SEQ ID NO: 18 - DNA, 23 bp, Artificial Sequence
  Synthetic forward primer
tgttggaaaatcatcaccttgct

SEQ ID NO: 19 - DNA, 22 bp, Artificial Sequence
  Synthetic reverse primer
ctgagtgtctgacgctgcttct

SEQ ID NO: 20 - DNA, 20 bp, Artificial Sequence
  Synthetic forward primer
catgcttgccacctcttgct

SEQ ID NO: 21 - DNA, 23 bp, Artificial Sequence
  Synthetic reverse primer
ccgtgactgcttgagttgtagct

SEQ ID NO: 22 - DNA, 20 bp, Artificial Sequence
  Synthetic forward primer
ttcgaggaggtcatgaagga

SEQ ID NO: 23 - DNA, 20 bp, Artificial Sequence
  Synthetic reverse primer
ttgctgttggcaggagatag

SEQ ID NO: 24 - DNA, 20 bp, Artificial Sequence
  Synthetic forward primer
ctaacccctgctcaaatcca

SEQ ID NO: 25 - DNA, 20 bp, Artificial Sequence
  Synthetic reverse primer
gccttgacaaatgtcggttt